(19) United States
(12) Patent Application Publication
(10) Pub. No.: US 2011/0142790 A1
(43) Pub. Date: Jun. 16, 2011

(54) Polyol Based - Bioceramic Composites

(75) Inventor: Qizhi Chen, Clayton (AU)

(73) Assignee: Monash University, Clayton (AU)

(21) Appl. No.: 12/952,596

(22) Filed: Nov. 23, 2010

Related U.S. Application Data
(60) Provisional application No. 61/264,589, filed on Nov. 25, 2009.

(51) Int. Cl. A61F 2/44; A61F 2/00; A61F 2/28; B32B 1/08; A61K 31/765; A61P 43/00

(52) U.S. Cl. 424/78.37; 523/113; 523/115; 428/34.1; 442/1

(57) ABSTRACT

Polyol-bioceramic composites are prepared by the reaction of a polyol and polycarboxylic acid in the presence of a bioceramic. Implantable medical devices fabricated at least in part with the crosslinked polyol-bioceramic composite materials are useful in a wide variety of applications.

### CROSS-REFERENCE TO RELATED APPLICATIONS

The present application claims priority from U.S. Provisional Application No. 61/264,589 filed 25 Nov. 2009, the content of which is incorporated herein by reference.

### FIELD OF THE INVENTION

The present invention relates to polyol based composite materials. In particular, the present invention relates to polyol-bioceramic based composite materials useful in tissue engineering.

### BACKGROUND OF THE INVENTION

Replacement of damaged or diseased body parts is an increasingly important part of medicine. For example, over 8 million surgical procedures are performed in the United States each year to treat the millions of Americans experiencing organ failure or tissue loss. Although procedures for organ transplantation and reconstructive surgery have the potential to dramatically improve quality of life, and in some cases save life, there are problems associated with them. These procedures often require either transplantation from a second surgical site, for example a skin and bone grafts, or organ donation from a healthy donor individual. Major problems with organ transplantation include the shortage of donor organs and the need for life-long administration of anti-rejection drugs. The problem with second site surgeries is that these procedures are associated with pain and in some cases, morbidity. Consequently, the science of tissue engineering has emerged with the goal of developing organs, tissues, and synthetic biomaterials which can be used to augment and/or replace traditional transplant technologies.

Collagen is the structural protein of connective tissues, such as skin (soft tissue) and bone (hard tissue). Although it has been described to be inelastic in contrast to elastin, another structural protein in connective tissue, collagen is actually elastic with elastic strain being 10-15% and the coefficient of restitution (resilience) being 90%, the same as that of elastin. A muscle fibre, i.e. muscle cell, is composed of three structural proteins: myosin, actin and titin. The reshaping ability of muscle fibre is provided by titin, a giant elastic protein with elastic strain being 150%.

The biocompatible and flexible polymers have been developed as an artificial substitute for collagen in connective tissue and muscular fibres in muscular tissue. To date, the biocompatible polymers most often utilised are thermoplastic polyesters, including poly(lactide acid) (PLA) and poly(glycolic acid) (PGA), as well as their copolymers (PLGA) or blends. To engineer connective and muscular tissues, which mostly work under dynamic loading conditions, such as in bone (constant and cyclic compression), heart and skeletal muscle (contraction and relaxation), the biomaterial should show long-term elasticity. These mechanical characteristics are impossible with thermoplastic polymers, because they undergo plastic (i.e. permanent) deformation almost immediately when loaded and their elongation at break is rather short, smaller than 3%.

Poly(polyol sebacate) (PPS) is a family of crosslinked elastomers recently developed for the applications of soft tissue engineering. Polyols are alcohols containing multiple hydroxyl groups. Glycerol, maltitol, sorbitol, xylitol and isomalt are some of the more common types. These types of polymers break down by simple hydrolysis to natural metabolisable by-products, and are therefore considered highly biocompatible. In vitro studies have indicated that degradation of poly(glycerol sebacate) (PGS) results in an acidic micro-environment. The acidic degradation products of other polymers, such as polyesters, lead to an inflammatory response and thus limit their ability to serve as a vehicle for cellular transplantation in most organ systems. It is envisaged that similar issues will occur during the degradation of PPS polymer systems.

The mechanical properties of PPS polymers may also change during in vivo degradation which can lead to a reduction in their mechanical properties.

Hence, there is a need for improved bioengineering materials which are more chemically and mechanically stable under in vivo conditions. It is desirable that the new family of composites will be biocompatible, elastic and tough, and will have a potential of wide applications in tissue engineering.

### SUMMARY OF THE INVENTION

In work leading up to the present invention, the inventors sought to develop improved biocompatible polyol composite systems which have broad applicability to tissue engineering.

In one aspect, the present invention provides a crosslinked polyol-bioceramic composite which comprises:
(A) a polymer matrix formed from the condensation reaction between (I) a polyol component containing at least three hydroxyl groups; (II) a polycarboxylic acid component containing at least two carboxylic groups; and
(B) at least one bioceramic material phase substantially homogeneously distributed throughout the polymer matrix;
wherein the amount bioceramic material in the composite being at least about 0.5% to about 20% by weight of the total weight of the composite.

In another aspect, the present invention provides a method of preparing a crosslinked polyol-bioceramic composite comprising the steps of:
(i) providing at least one polyol component containing at least three hydroxyl groups;
(ii) providing at least one polycarboxylic acid component containing at least two carboxylic acid;
(iii) partially reacting the polyol with the polycarboxylic acid to form a prepolymer solution;
(iv) substantially homogeneously distributing at least one bioceramic material throughout the prepolymer solution; and
(v) subjecting the prepolymer solution of step (iv) to further reaction conditions to introduce further crosslinking to form the crosslinked polyol-bioceramic composite.

In another aspect, the present invention provides a crosslinked polyol-bioceramic scaffold composite comprising
(A) a porous bioceramic foam formed from at least one bioceramic material; and
(B) a polyol polymer matrix wherein the polyol polymer matrix is formed in situ in the foam by the condensation reaction of (I) a polyol component containing at least three hydroxyl groups; (II) a polycarboxylic acid component containing at least two carboxylic groups;
   wherein the amount bioceramic material in the polyol-bioceramic scaffold composite being at least about 50% to about 70% by weight of the total weight of the polyol-bioceramic scaffold composite.

### BRIEF DESCRIPTION OF THE ACCOMPANYING DRAWINGS

FIG. 1: Illustrates the pH values of culture medium after incubation with PGS and Poly-DL-lactic acid (PDLLA).

FIG. 2: Illustrates the pH values of culture medium after incubation with PGS-BG composites.

FIG. 3: Illustrates cell numbers after cultured with extracts of materials for 2 days.

FIG. 4: Illustrates the dead cells during the 2-day culturing in extracts of materials. The differences of PGS-15% BG vs other samples are significant (p<0.01). No significant differences in cell death were revealed among other samples.

FIG. 5: Illustrates the percentage dead/live cells during the 2-day culturing in extracts of materials. The differences of PGS-15 wt % BG vs other samples are significant (p<0.01). No significant differences in cell death were revealed among other samples.

FIG. 6: Illustrates Young's modulus of PGS-BG composite materials vs weight percentage of BG.

FIG. 7: Illustrates Ultimate tensile strength of PGS-BG composite materials vs weight percentage of BG.

FIG. 8: illustrates the Elongation at rupture of PGS-BG composite materials vs percentage of BG.

FIG. 9: Illustrates (a) Plot of Stress (MPa) vs Strain for pure poly(glycerol sebacate) (PGS); (b) Plot of Stress (MPa) vs Strain with PGS-10 wt % BG composite.

FIG. 10: Illustrates plot of ultimate tensile strength (UTS, MPa) for pure PGS, PGS-5% HA and PGS-10% HA.

FIG. 11: Illustrates Young's modulus (MPa) for pure PGS, PGS-5 wt % HA and PGS-10 wt % HA.

FIG. 12: Strain at break for pure PGS, PGS-5 wt % HA and PGS-10 wt % HA.

FIG. 13: Illustrates pH measurement of medium soaked with PXS, and PXS-BG composite at 2%, 5% and 10% wt % BG.

FIG. 14: Illustrates elongation at rupture for PXS and PXS-BG at 2%, 5% and 10% wt % BG.

FIG. 15: Ultimate tensile strength (UTS, MPa) of PXS and PXS-BG at 2%, 5% and 10% wt % BG.

FIG. 16: Young's modulus of PXS and PXS-BG at 2%, 5% and 10% wt % BG.

FIG. 17: (a)-(b) Porous structure of Bioglass-derived ceramic scaffolds before and after being coated with poly(glycerol sebacate), respectively. (c)-(d) Microstructure of the struts before and after coating of poly(glycerol sebacate), respectively.

FIG. 18: Illustrates the compressive mechanical strengths of porous network with or without PGS coatings (coating of PGS was followed by a crosslink treatment).

FIG. 19: Illustrates the FTIR spectrum of Bioglass®, pure poly(glycerol sebacate) (PGS) and Bioglass® network coated with PGS and treated for crosslink. The peak at 1573 cm⁻¹ in the spectrum of Bioglass®-PGS is the vibration band of sodium carboxylate group.

FIG. 20: Illustrates the XRD spectra of 45S5 Bioglass®-ceramic foams (a) sintered at 1000° C. for 1 hr and (b) coated with poly(glycerol sebacate); which were immersed in simulated body fluid for 3, 7 and 30 days. All spectra were obtained using 0.1 g powder. The major peaks of Na₂Ca₂Si₃O₉ phase and hydroxyapatite are marked by ∇ and , respectively.

FIG. 21: TEM observation of Bioglass®-ceramic-PGS composite heat treated at 120° C. for 3 days and then soaked in a tissue culture medium. (a) Unbroken particles with (b) dissolution at surface were the dominant morphology after soaking for 3 days. (c-d) Nanosized particles were evident after incubation for 14 days and longer (TEM images from the samples of 30 days were shown here). (c) Clusters of nanoparticles derived from original micro-sized particles; (d) the nanoparticles well embedded in the polymer matrix.

FIG. 22: Raw data of compressive strength of Bioglass®-PGS scaffolds treated at 120° C. for 3 days, which was soaked in a tissue culture medium for up to 2 months.

FIG. 23: Schematic healing rate of growing bone (C1), degradation kinetics of an ideal scaffold (C2) and typical degradation kinetics of resorbable but mechanically fragile materials (C3 & C4) and of inert (mechanically strong)) materials (C5).

FIG. 24: Illustrates the SNL cell proliferation kinetics measured by the AlamarBlue™ technique. The initial plating density was 5000 cells/ml each well in a 48-well plate (n=3). Overall, the differences between any two of the three groups were not significant (p>0.05).

FIG. 25: Acidity of culture media during incubation with PGS and its nanoBioglass® composites. Data of day 0 were measured after incubation for 1 h. The acidity of medium only and for medium plus composites were not significantly different (p>0.05), but the differences in the acidity of the medium only and the medium plus each of the PGS specimens were significantly different (p<0.001 or 0.01).

FIG. 26: Tensile stress-strain curves of (a) pure PGS, (b) 2 wt % and (c) 5 wt % nanoBioglass-filled PGS composites before and after soaking in tissue culture medium at 37° C. under 5% CO₂ atmosphere. The materials were crosslinked under vacuum at 120° C. for 2 days.

FIG. 27: Young's modulus of PGS and nanoBioglass-filled composites before and after incubation in tissue culture medium at 37° C. under 5% CO₂ atmosphere.

FIG. 28: Cytotoxicity of different test materials, detected by measuring the release of lactate dehydrogenase (LDH).

FIG. 29: Representative distribution of halloysite nanotubes in the PGS/halloysite composites of (a) 3, (b) 5 and (c) 10 wt % concentrations.

FIG. 30: pH values of halloysite water slurries of 0, 1, 3, 5, 10 and 20 wt % clay.

### DETAILED DESCRIPTION OF THE INVENTION

The new composites possess several advantages over thermoplastics and pure PPS. The PPS-based bioceramics-reinforced composites can buffer microanatomic environment and maintain its pH value close to the normal physiological condition. The composites have a more predictable biocompatibility than pure PPS, and their biocompatibility is comparable to the clinically applied polymer Poly-DL-lactic acid (PDLLA) in terms of cytotoxicity and cell proliferation. The PPS-10 wt % BG composites are tougher than thermoplastics/related composites and pure PPS. Depending on the formulation used to prepare the composite, the composites may be made to be as soft and flexible as soft tissues. The composites could provide a stable and reliable mechanical function over the initial period of implantation.

The amount of bioceramic material used in the preparation of the composites of the present invention may be at least about 5% to about 15% by weight of the total weight of the composite. Preferably, at least about 10% by weight of the total weight of the composite.

The polyol component used to prepare the inventive composites may be selected from the group comprising glycerol, erythritol, threitol, ribitol, arabinitol, xylitol, allitol, alritol, galactitol, sorbitol, mannitol, iditol and malitol. Preferably the polyol used is glycerol, maltitol, sorbitol, xylitol or isomalt. More preferably, the polyol component is glycerol.

The polycarboxylic acid component may be selected from the group comprising a metabolite, an aldaric acid, an alkanedioic acid, an alkenedioic acid, or an amino acid, or a derivative or salt thereof.

In one embodiment, the polycarboxylic acid component is an aldaric acid selected from the group comprising 2-hydroxy-malonic acid, tartaric acid, ribaric acid, arabanaric acid, xylaric acid, allaric acid, altraric acid, galacteric acid, glucaric acid, or mannaric acid, or a derivative or salt thereof.

In another embodiment, the polycarboxylic acid component is a metabolite selected from the group comprising succinic acid, fumaric acid, α-ketoglutaric acid, oxaloacetic acid, malic acid, oxalosuccinic acid, isocitric acid, cis-aconitic acid, or citric acid, or a derivative or salt thereof.

In another embodiment, the polycarboxylic acid component is an alkanedioic acid selected from the group comprising dimercaptosuccinic acid, oxalic acid, malonic acid, succinic acid, glutaric acid, adipic acid, pimelic acid, suberic acid, azelaic acid, or sebacic acid, or a derivative or salt thereof. Preferably, the alkanedioic acid is sebacic acid, or a derivative or salt thereof.

In another embodiment, the polycarboxylic acid component is an alkenedioic acid selected from the group comprising fumaric acid, maleic acid, glutaconic acid, itaconic acid, mesaconic acid, or traumatic acid, or a derivative or salt thereof.

In another embodiment, the polycarboxylic acid component is an amino acid selected from the group comprising aspartic acid or glutamic acid, or a derivative or salt thereof.

The bioceramic used in the preparation of the composites of the present invention may be selected from the group comprising alumina, zirconia, apatites, calcium phosphates, silica based glasses, and bioactive glass ceramics and combinations and modified foams.

In one embodiment, the bioceramic is an apatite. The apatite may be selected from the group comprising hydroxyapatite (Ca₁₀(PO₄)₆(OH)₂), floroapatite (Ca₁₀(PO₄)₆F₂), chlorapatite (Ca₅Cl(PO₄)₃), carbonate apatide (Ca₁₀H₂(PO₄)₆-5H₂O)) and combinations and modified forms. Preferably the apatite is hydroxyapatite.

In another embodiment, the bioceramic may be a bioactive glass. With this embodiment, the bioactive glass may be selected from the group comprising 45S5, 58S, S53P4, S70C30 and combinations and modified forms. Preferably, the bioactive glass is 45S5, which is commonly referred to as Bioglass®.

In another embodiment, the bioceramic may be an aluminosilicate. In one embodiment, the aluminosilicate is a nanotubular halloysite which is a 1:1 aluminosilicate clay mineral with the empirical formula Al₂Si₂O₅(OH)₄.

The polyol-bioceramic composite of the present invention may be used to treat a disease, condition, or disorder from which a subject is suffering.

The crosslinked polyol-bioceramic composite of the present invention may be adapted and constructed to have a shape selected from particles, tube, sphere, strand, coilend strand, capillary network, film, fibre, mesh and sheet.

The crosslinked polyol-bioceramic composite of the present invention may be used as a tissue engineering construct, as a nerve conduit, as a mesh to be used in surgical abdominal hernia repair, or in intervertebrate disc repair.

Polyol-based polymers useful in the preparation of the inventive composite materials are described in, for example, WO 2008/144514 Entitled “Polyol-based polymers”, the contents of which are hereinbefore incorporated by reference. Other examples of suitable polyol polymer systems are described, in for example, Biomaterials 29 (2008) 4726-4735 Entitled” Biodegradable poly(polyol sebacate polymers), the contents of which are hereinbefore incorporated by reference.

Bioceramics can include any ceramic material that is compatible with the human body with reactive hydroxyl or amine groups. More generally, bioceramic materials can include any type of compatible inorganic material or inorganic/organic hybrid material with reactive hydroxyl or amine groups. Bioceramic materials can include, but are not limited to, alumina, zirconia, apatites, calcium phosphates, silica based glasses, or glass ceramics, and pyrolytic carbons. Bioceramic materials can be bioabsorbable and/or active. A bioceramic is active if it actively takes part in physiological processes. A bioceramic material can also be “inert,” meaning that the material does not absorb or degrade under physiological conditions of the human body and does not actively take part in physiological processes.

Illustrative examples of apatites and other calcium phosphates, include, but are not limited hydroxyapatite (Ca₁₀(PO₄)₆(OH)₂), floroapatite (Ca₁₀(PO₄)₆F₂), carbonate apatide (Ca₁₀H₂(PO₄)₆-5H₂O)), calcium phosphate, Mg-substituted tricalcium phosphate, dicalcium phosphate, tricalcium phosphate (Ca₃(PO₄)₂), octacalcium phosphate (Ca₈H₂(PO₄)₆-5H₂O), amorphous calcium phosphate, calcium pyrophosphate (Ca₂P₂O₇-2H₂O), tetracalcium phosphate (Ca₄P₂O₉), carbonate hydroxyapatite and dicalcium phosphate dehydrate (CaHPO₄-2H₂O).

The calcium phosphate may be selected from the group comprising Cerap Atite®, Synatite®, Biosorb®, Calciresorb®, Chronos®, Biosel®, Ceraform®, Eurocer®, Mbcp®, Hatric®, Tribone 80®, Triosite®, Tricos® and mixtures thereof

The term bioceramics can also include bioactive glasses that are bioactive glass ceramics composed of compounds such as SiO₂, Na₂O, CaO, and P₂O₅. For example, a commercially available bioactive glass, Bioglass®, is derived from certain compositions of SiO₂—Na₂O—K₂O—CaO—MgO—P₂O₅ systems. Some commercially available bioactive glasses include, but are not limited to:

45S5: 46.1 mol % SiO₂, 26.9 mol % CaO, 24.4 mol % Na₂O and 2.5 mol % P₂O₅;

58S: 60 mol % SiO₂, 36 mol % CaO, and 4 mol % P₂O₅; and

S70C30: 70 mol % SiO2, 30 mol % CaO.

A common characteristic of bioactive glasses and ceramics is a time-dependent kinetic modification of the surface that occurs upon implantation. The surface forms a biologically active hydroxyl carbonate apatite (HCA) layer which provides the bonding interface with tissues. The HCA phase that forms on bioactive implants is chemically and structurally equivalent to the mineral phase in bone providing interfacial bonding. An overview of different bioactive glass compositions and their corresponding bioactivities is given in, for example, Hench, L L., “Bioceramics: from concept to clinic”, J. Am. Ceram. Soc, 1991, 74, 1487-510, the contents of which are hereinbefore incorporated by reference.

Various sizes of the bioceramic particles may be used in the composite. For example, the bioceramic particles can include, but are not limited to, nanoparticles and/or micro particles. A nanoparticle refers to a particle with a characteristic length (e.g., diameter) in the range of about 1 nm to about 1,000 nm. A micro particle refers to a particle with a characteristic length in the range of greater than 1,000 nm and less than about 10 micrometers. Additionally, bioceramic particles can be of various shapes, including but not limited to, spheres and fibers.

Polyol composite materials with high levels of bioceramic: An alternative embodiment of the present invention allows the fabrication of biocomposites with high levels of bioceramic. A new composite scaffold has been engineered from an elastomer poly(glycerol sebacate) (PGS) and BG. In addition to a bone-bonding ability and excellent biocompatibility, the new composite scaffold exhibits unique mechanical properties that have never been reported for any existing scaffolds. First, it possesses a predictable mechanical strength that is close to the theoretical strength limit. Second it has a mechanically steady state over a period of degradation in a physiological environment while the structure of composite material is disrupted. The second feature is of great importance to tissue engineering that requires a mechanically steady state post implantation before the onset of rapid degradation kinetics.

In certain embodiments, the inventive polyol-bioceramic composite is a component of a biomedical device or implant. In certain embodiments, the inventive polyol-bioceramic is a polymer film or coating on an implant. In certain embodiments, the inventive polymer is an implant. In certain embodiments, the inventive polymer implant is a polymer matrix.

In one embodiment, the inventive polyol-bioceramic composite is surgically implanted or injected into a subject on or near diseased or damaged tissue. In certain embodiments, the inventive polymer implant aids in the in-growth of surrounding healthy tissue to the diseased area.

The polyol-bioceramic composite may be produced in different foams, depending upon the intended use and purpose. Suitable forms include solid, putty, and paste, depending on the degree of crosslinking of the polyol. If the polyol-bioceramic composite is in solid form, it may be, for example, a shaped or unshaped solid, it may be a pre-formed solid, it may be a frame or a lattice, or another solid form. The solid form may be very stiff, stiff, slightly flexible, soft, rubbery, or other. The polyol-bioceramic composite may be a putty. If in putty form, it may be anywhere from a dense or thin putty. The polyol-bioceramic composite may be a paste. If a paste, it may be anywhere from a thick to a thin paste.

In one embodiment, the bioceramic may be formed into a porous scaffold prior to the addition of the polyol components and then crosslinked to form a polyol-bioceramic composite. This type of preparation is particularly suitable for producing composites with high bioceramic loading.

The present invention provides a method of making an inventive polymer composite comprising the steps of:
(i) providing a polyol;
(ii) providing a polycarboxylic acid, or derivative thereof;
(iii) providing a bioceramic and
(iv) reacting the polyol with the polycarboxylic acid in the presence of the biocermaic to form a polymer composite.

A person skilled in the art will appreciate that a wide variety of reaction conditions may be employed to promote the above transformation, therefore, a wide variety of reaction conditions are envisioned; see generally, March's Advanced Organic Chemistry: Reactions, Mechanisms, and Structure, M. B. Smith and J. March, 5th Edition, John Wiley & Sons, 2001, and Comprehensive Organic Transformations, R. C. Larock, 2nd Edition, John Wiley & Sons, 1999, the entirety of both of which are incorporated herein by reference.

In certain embodiments, the reaction of step (iv) is a condensation reaction {e.g., reaction between a carboxylic acid or derivative thereof and an alcohol, with the extrusion of water, an alcohol by-product, or a suitable leaving group). In certain embodiments, the reaction of step (iv) further comprises the application of heat. In certain embodiments, the reaction of step (iii) comprises heating the polyol and the polycarboxylic acid to a temperature of at least 50° C. In certain embodiments, the reaction is heated to a temperature of at least 60° C., 70° C., 80° C., 90° C., 100° C., 110° C., 120° C., 125° C., 130° C., 135° C., 140° C., 145° C., 150° C., 155° C., 160° C., 165° C., or 170° C.

In certain embodiments, the reaction of step (iii) further comprises conducting the reaction under reduced pressure.

Optionally, other components or additives may be added to the polyol-bioceramic composite. These additives may be added for various reasons. For example, additives may be added to increase biocompatibility, to decrease the possibility of rejection, to decrease the risk of infection, to increase the rate of natural bone growth in the bioceramic, or to increase the rate of natural cell growth near the implant. Additives may also be added to change or enhance some of the properties of the bioceramic. For example, the bioceramic may include growth factors, cells, other materials and elements, curing or hardening components, and other possible additives.

In a particular embodiment, the present invention provides a poly(glycerol sebacate)-bioglass composite which comprises:
(A) a polymer matrix formed from the condensation reaction between (I) glycerol; (II) sebacic acid; and
(B) Bioglass® substantially homogeneously distributed throughout the polymer matrix;
   wherein the amount Bioglass® in the composite being at least about 0.5% to about 20% by weight of the total weight of the composite.

The invention is illustrated by the following non-limiting examples.

Materials and Methods: 45S5 Bioglass® powder was purchased from Novabone®Product, with particle size being ˜5 μm on average. This glass has a composition of 45 wt. % SiO₂, 24.5 wt. % CaO, 24.5 wt. % Na₂O and 6 wt. % P₂O₅. Unless stated otherwise, all other materials were obtained from Sigma.

Statistics: All experiments were run with five samples, and the data are represented as mean±SE. Statistical difference was analysed using one-way analysis of variance (ANOVA) with Tukey's post-hoc test, and a p value of <0.05 was considered significant.

### EXAMPLE 1

### Synthesis of poly(glycerol sebacate) (PGS) prepolymer

A PGS pre-polymer was synthesized by polycondensation of 1:1 M ratio of the triol, glycerol (purity 99%) and the diacid, sebacic acid (purity 99%). The polycondensation reaction was initially carried out at 125° C. for 24 h under nitrogen gas—at this stage, the reaction was incomplete and the pre-polymer was still ungelled and could be dissolved in THF to produce a 50 wt/v % solution, as illustrates in Scheme 1.

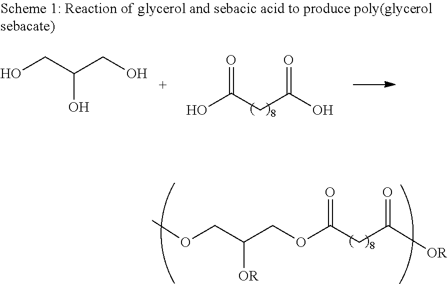

### EXAMPLE 2

### PGS-Bioglass® (BG) Composite

Four percentages (0, 1, 5, 10 and 15 wt. %) of 45S5 Bioglass® were added to a 50 wt. % solution of the PGS pre-polymer in tetrahydrofuran (THF) solution and magnetically stirred thoroughly. It was noticed that after the addition of the Bioglass® to the pre-polymer solution, the fluid's viscosity was remarkably increased and this is partly due to reaction between the PGS and filler. The THF solution/slurry was cast onto glass slides and the THF evaporated at ambient conditions to produce ˜1 mm thick sheet of PGS pre-polymer. Finally the cast sheet was further polymerized at 125° C. for an additional 48 h under vacuum to increase the crosslink density of the final material. After soaking in deionizer water for 5 hours, the sheets could be easily peeled off.

### EXAMPLE 3

### Acidity Testing of PGS-Bioglass® Composite

Acidity testing was carried out by utilizing a small piece of the polymer samples, weighing approximately 0.4 g. These miniature pieces were sterilized in a 70% alcohol/deionised water solution. After allowing the samples to dry for 2 hours, each sample was then soaked in 4 mL of Dulbecco's Modified Eagle's Medium (DMEM) tissue culture medium and placed in a sterilised 15 mL centrifuge tubes. These tubes were then placed in an incubator at 37° C. under 5% CO₂ atmosphere in order to simulate similar conditions that you would find in the human body. The acidity measurements were carried out by using a pH meter while the samples were still inside the incubator at the prescribed environmental conditions. On day 0, the first acidity measurement was made after incubation of the samples had preceded for 4 hours when the conditions in the incubator matched 37° C. and 5% CO₂ atmosphere. These measurements were repeated 24 and 48 hours later on day 1 and day 2 respectively to determine the pH levels over the testing period.

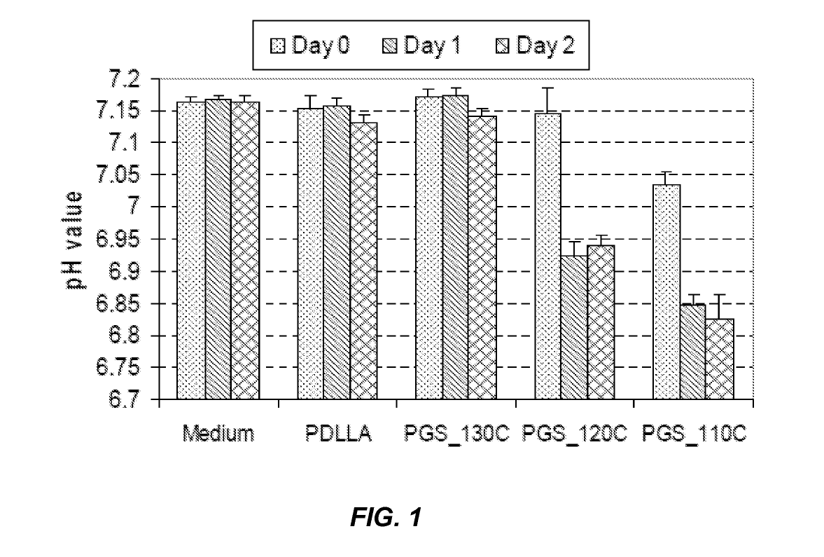

FIG. 1 demonstrates the comparative pH values of the culture environment, PDLLA and PGS polymer samples. Compared with clinically applied degradable polyester PDLLA, PGS crosslinked at 130° C. did not introduce considerable acidity during its degradation, whereas PGS crosslinked at 120 and 110° C. caused significant decreases in the pH values of the culture medium after one-day incubation. Unfortunately, the PGS synthesised at 130° C. were fully crosslinked and brittle and have little potential to produce tough (strong and elastic) composites.

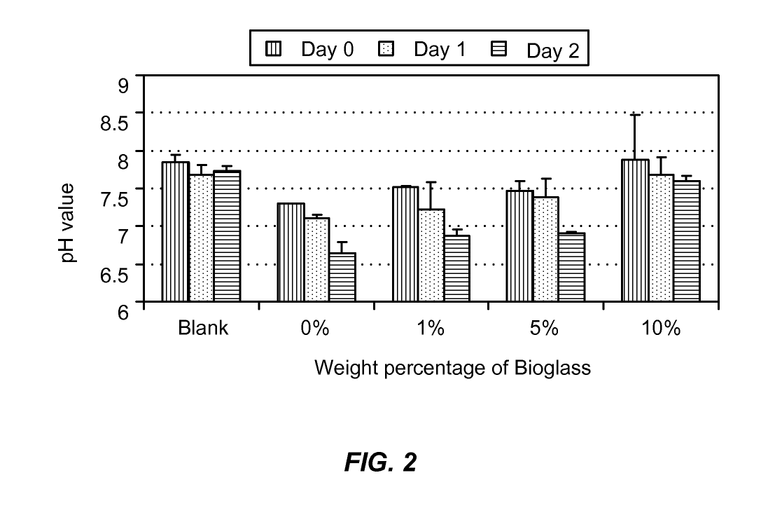

FIG. 2 illustrates the pH values of culture medium when incubated with PGS-BG composite samples. It was revealed that the pH value of the culture microenvironment could be maintained at the normal (nearly neutral) level of the body with 5 and 10 wt % BG-PGS composites, and shows an improvement in pH stability compared to even for the PGS-1 wt % BG composite.

### EXAMPLE 4

### Cytocompatibility In Vitro (ISO 10993)

Cytocompatibility study was performed according to the standard cytotoxicity assessment set by International Standardization Organization (ISO 10993). Extracts for tissue culture were prepared by placing 0.4 g of each material in 2 ml samples of cell culture medium (DMEM supplemented with 10% Fetal Calf Serum (FCS), 1% L-glutamine and 0.5% penicillin/streptomycin) for 24 h at 37° C./5% CO₂ in culture incubator. Poly(D,L-lactic acid) (PDLLA, from PURASORB®, Netherlands) was used as the material control (PDLLA was sterilized by 70% alcohol/deionized water solution at ambient conditions), and 2 ml of cell culture medium alonewas the negative control. Prior to exposure of cells to these extracts, SNL mouse fibroblasts (Mutant Mouse Regional Resource Centers, University of California Davies, USA) were seeded in standard media at a density of approximately 2000 cells/well in 96 well tissue culture treated plates (Falcon, BD Bioscience, North Ryde, Australia), under standard incubation conditions (37° C. and 5% CO₂), with medium changed every second day. When the cell monolayers had reached 80% confluence (around day 4), the medium in each well was entirely replaced with 0.2 ml of extract media samples (medium preexposed to material) or control media (material control=medium pre-exposed to PDLLA; negative control=medium only). All cultures were then allowed to proceed for 2 days.

At the end of the incubation period, spent culture media were collected and the degree of cell death was determined by measurement of lactate dehydrogenase (LDH) levels, as released into the culture media (“RELEASED LDH”), using a commercial kit (SigmaeAldrich TOX-7) as we have described previously. Finally, each well containing living cells was filled with 0.2 ml fresh cell culture medium and cells were lysed using the solution TOX-7. These lysates were then used to determine the cellular LDH content, which equates to the number of living cells per well (“TOTAL LDH”). The overall LDH level was determined by measuring the absorbance of the supernatant from the centrifuged medium at 490 nm (after subtraction for background absorbance at 690 nm) using a multiwell plate format UVevis spectrophotometer (Thermo Scientific). The absorbance results of LDH were converted to the number of cells according to a linear standard curve (not shown).

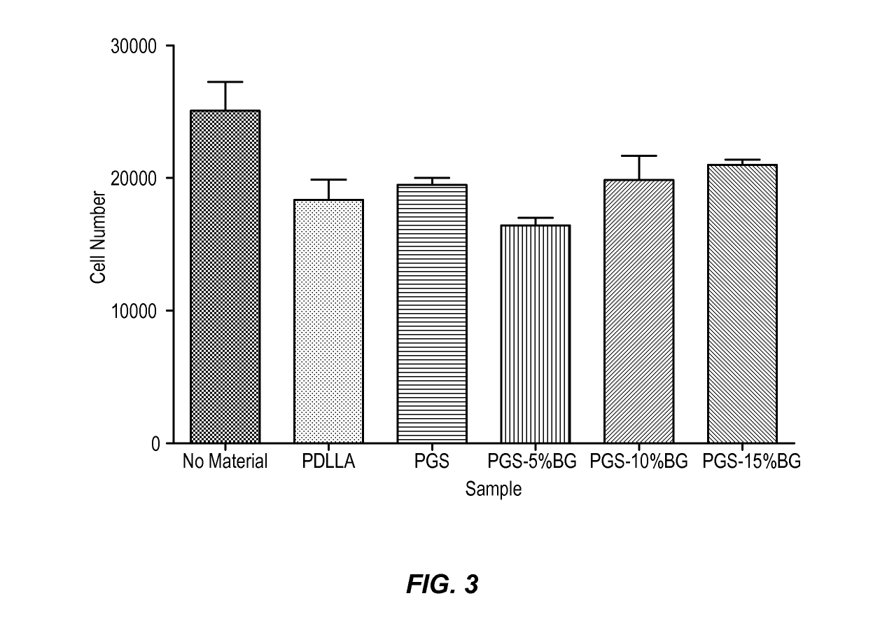

FIG. 3 shows the number of living cells after cultured with extracts of materials for 2 days for blank, PDLLA, PGS, PGS-5 wt % BG, PGS-10 wt % BG. It was observed that cells proliferated well on all materials (test and control), with no significant difference in cell numbers (p>0.05). Compared with tissue culture plate (i.e. no test and control materials), the cell numbers were significantly reduced when cultured with extracts of PDLLA and PGS-5 wt % BG samples.

No Material vs PDLLA (p<0.05), No Material vs PGS-5 wt % BG (p<0.01). Differences between any other two groups were not significant (p>0.05).

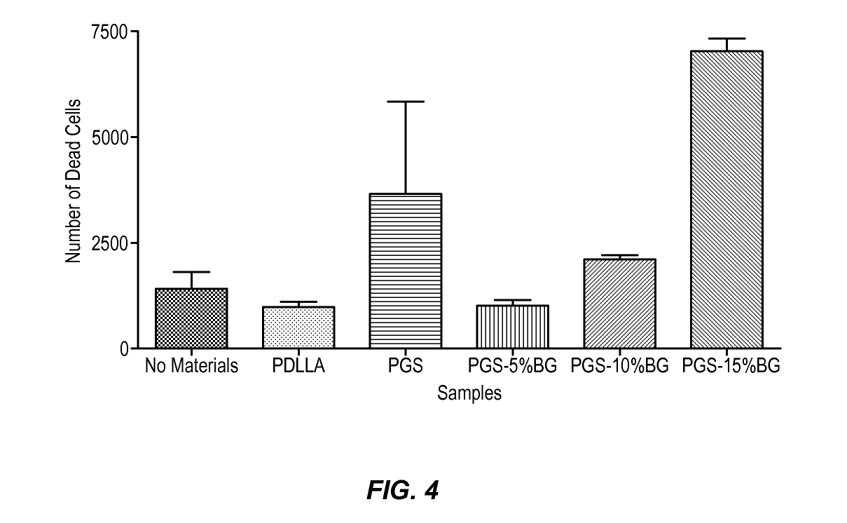

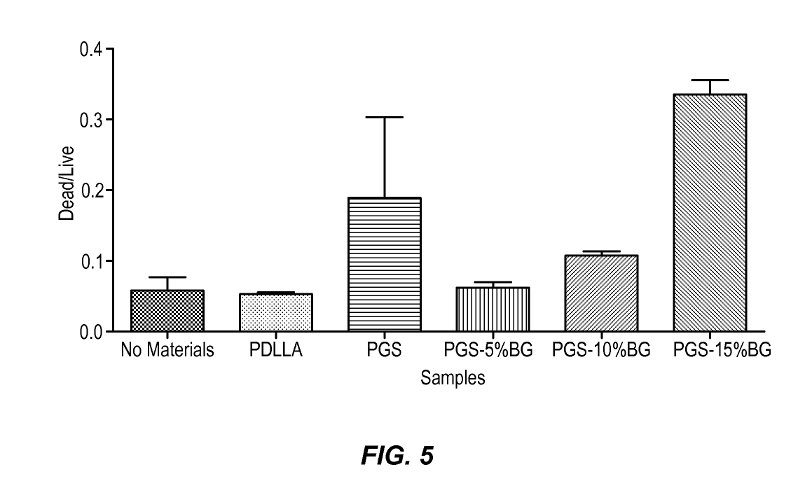

FIGS. 4 and 5 illustrate the number of dead cells and the percentages of dead/live cells. PGS-15 wt % BG samples showed significant cytotoxicity, probably because of the overshoot of pH. Too alkaline environment could be the reason. Although pure PGS did not show significant difference statistically, it must be mentioned that there was a large variation from one sample to another, and this indicated the inhomogeneity of this material, whereas PGS-BG materials are much more predictable with small variations. In conclusion, PGS doped with 5-10 wt % BG showed the best biocompatibility, compared with pure PGS and PGS-15 wt % BG materials.

### EXAMPLE 5

### Mechanical Properties of PGS-BG Composites

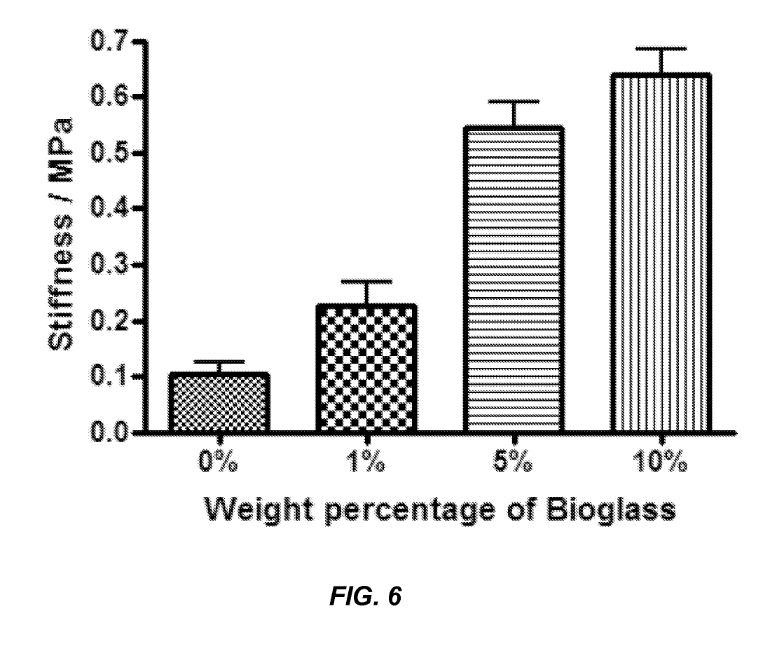

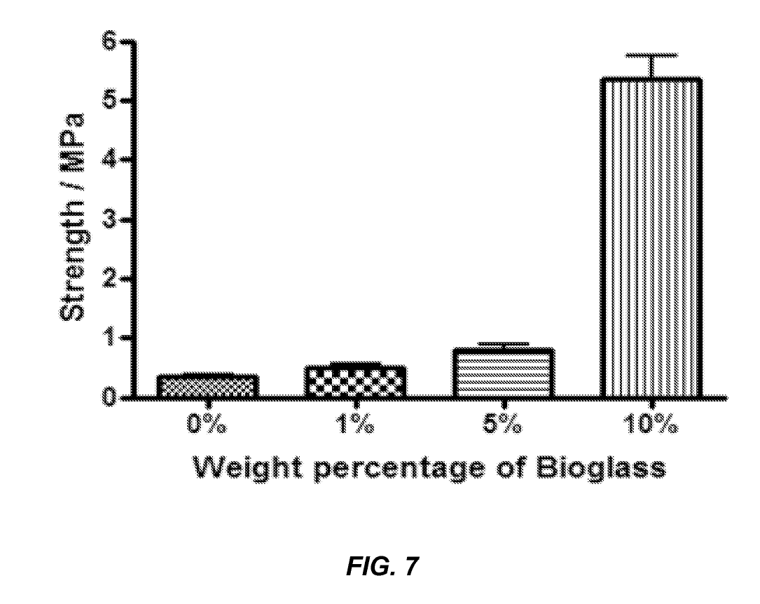

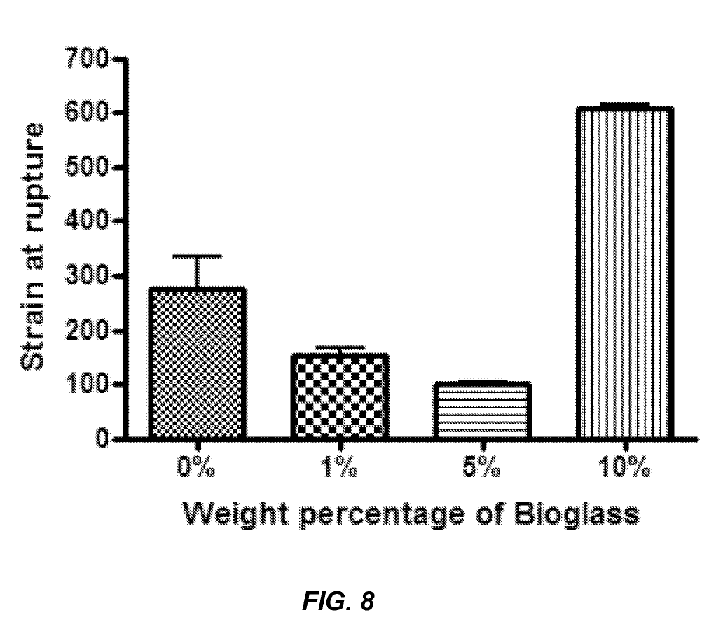

Mechanical properties for each of the composites were determined including ultimate tensile strength (UTS), Young's modulus and strain at rupture, as shown in FIGS. 6 to 8. The UTS and young's modulus increased with the percentage of added BG. The strain at rupture decreased first with the increasing of BG concentration. However, it increased significantly in PGS-10 wt % BG, changing from less than 300% in pure PGS to larger than 600% in PGS-10 wt % BG.

The observed increase in strength is surprising as it is far and beyond what you would expect from merely the introduction of 10 wt % BG.

### EXAMPLE 6

### Degradation of PGS-BG Composites

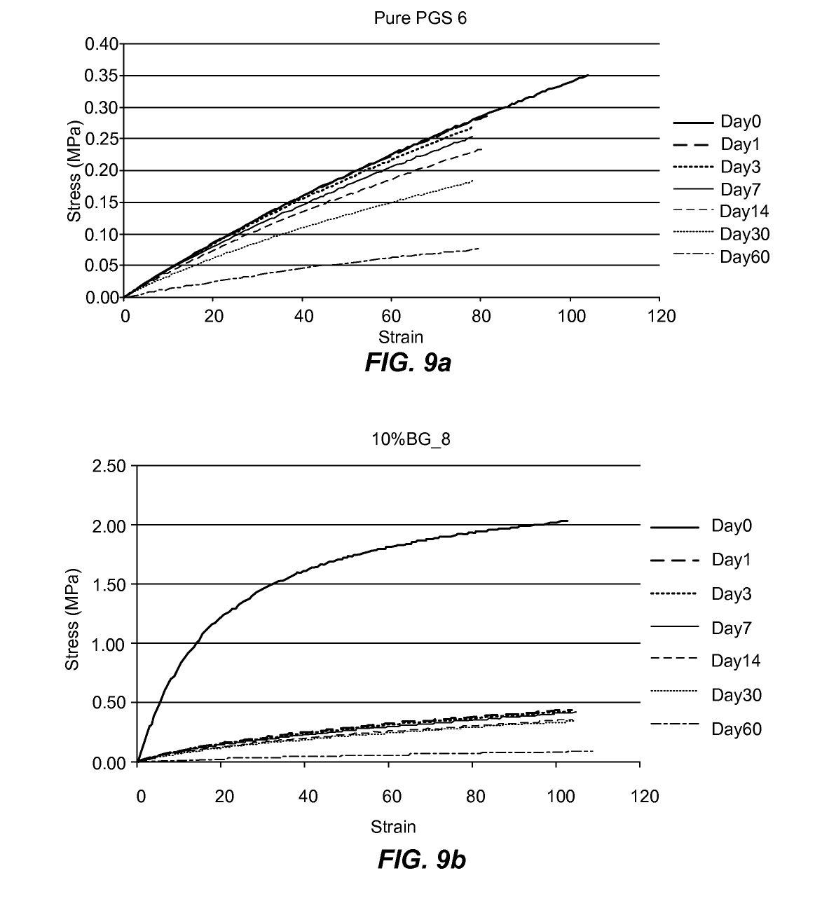

The mechanical properties of these materials during degrading were determined in vitro. FIGS. 9a and 9b demonstrate the change of stress-strain curves of pure PGS and PGS-BG composite over incubation time. It can be seen that after one day soaking the mechanical strength of the composites immediately dropped to the level of pure PGS, and then remain relative stable. This is a very useful mechanical behaviour. In many applications to soft tissue engineering, the addition of BG is expected to buffer the pH of a physiological environment and provide a stable mechanical support over the initial implantation period. An implant that is mechanically too strong to match soft tissue could cause significant pain for the patients.

The results for the strain experienced by the samples are surprising. The common belief is that mixing a polymer with a ceramic is that the composite would have properties that lie between the two materials. The polymer component allows for large amount of elongation as the chains stretch when the material is under tension. However, the ceramic Bioglass component does not have the same ability to extend when under tension. Thus, one would assume that the overall elongation of the test samples would decrease as more Bioglass is added. This theory does prove accurate for the first three polymer mixes. As demonstrated, the elongation decreases when more Bioglass is added. However, when 10 weight percent of Bioglass is added, this theory becomes in consistent with the observed results. The 10 wt % samples have a far larger ability to strain that the polymer alone. This seems to indicate that there is some new form of interaction between the two materials that takes place in the microstructure when the weight percentage of Bioglass in the polymer reaches a significant amount.

The stiffness of the polymer changes dramatically when different amounts of Bioglass is added to the polymer matrix. The stiffness of the polymer increases with increasing presence of Bioglass, until around 10 weight percent is added. After this point, the stiffness begins to reduce again, as can be seen by the decline in stiffness from 10 to 15 weight percentage. Typically, when a ceramic is added to the polymer matrix, the overall stiffness, max strain and stress required to cause fracture do not all increase simultaneously. This surprising property of the composites of the present invention mean that the properties of the composite may be tailored to a particular application.

### EXAMPLE 7

### PGS-Hydroxyapatite (HA) Composite

A series of PGS-hydroxyapatite composites were prepared by mixing hydroxyapatite (HA) powder into the PGS prepolymer solution prepared in Example 1 to produce 1, 5, 10 and 15 wt % percentage PGS-BG composite. As a reference, a PGS polymer was prepared which contained 0% wt % HA. The slurries were then vigorously stirred for at least 1 hour and the resulting solution cast onto glass slides to produce sheet materials. The cast slurry was then dried at ambient condition for 24 hours and under vacuum in oven for another 24 hours. Finally, the materials were then treated at 120-130° C. for 2-5 days to crosslink the PGS. After soaking in deionizer water for 5 hours, the sheets could be easily peeled off.

### EXAMPLE 8

### Mechanical Properties of PGS-HA Composites

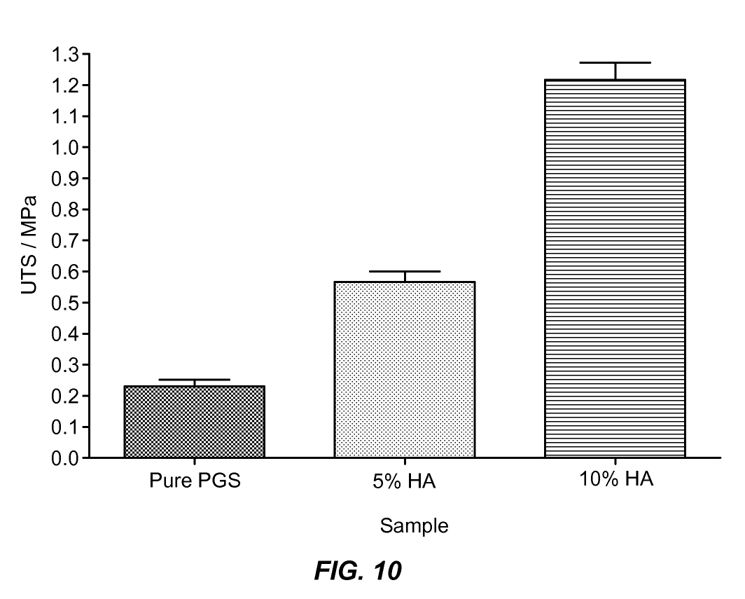

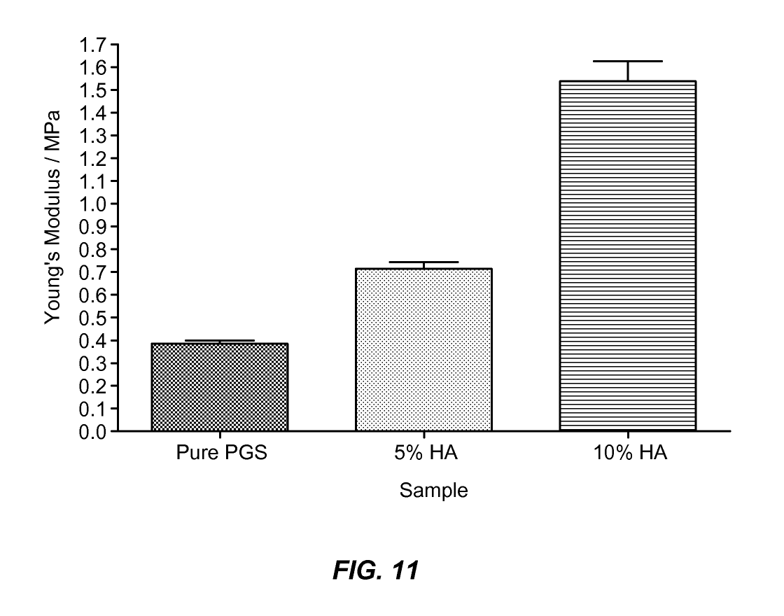

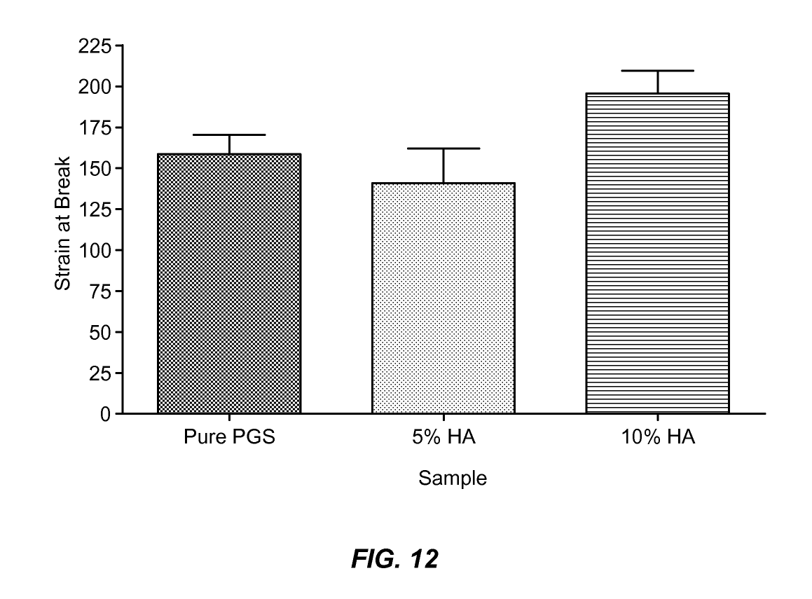

Mechanical properties for each of the PGS-HA composites were determined including ultimate tensile strength (UTS), Young's modulus and strain at rupture, as shown in FIGS. 10 to 12. The UTS and young's modulus increased with the percentage of added HA. The strain at break/rupture decreased first at 5 wt % HA then increased significantly in PGS-10 wt % HA, changing from less than 150% in pure PGS in this system to larger than 200% in PGS-10 wt % HA. The qualitative strength is potentially sintered

The above unusual increment in strain at rupture by second fillers has been reported in elastomers filled with nano-particles, but not with micro particles. The particles size of the present bioceramics is 1-5 microns.

### EXAMPLE 9

### Synthesis of poly(xylitol sebacate) (PXS) prepolymer

PXS prepolymer was synthesized by polycondensation of xylitol and sebacic acid at 120-130° C. under argon for 12-24 hr. The prepolymer was then dissolved in tetrahvdrofuran (THF) to produce a 50 wt/v % solution.

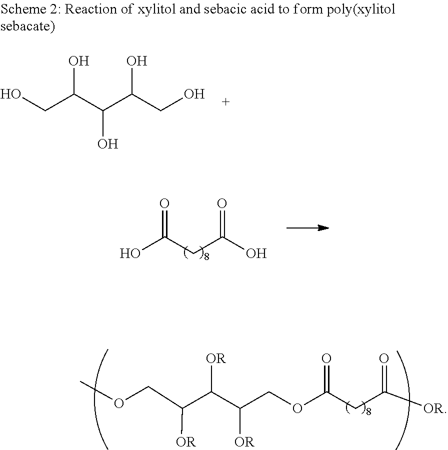

### EXAMPLE 10

### PXS-Bioglass (BG) Composite

A series of PXS-bioceramic composites were prepared by mixing BG powder into the PXS prepolymer solution prepared in Example 9 to produce 2, 5, 10 and 15 wt % percentage PGS-BG composites. As a reference, a PXS polymer was prepared which contained 0% wt % BG. The slurries were then vigorously stirred for at least 1 hour and the resulting solution cast onto glass slides to produce sheet materials. The cast slurry was then dried at ambient condition for 24 hours and under vacuum in oven for another 24 hours. Finally, the materials were treated at 120-130° C. for 2-5 days to crosslink the PXS. After soaking in deionizer water for 5 hours, the sheets could be easily peeled off.

### EXAMPLE 11

### pH Testing of PXS-BG Composites

Acidity testing was carried out as described above on small pieces of the polymer samples, weighing approximately 0.4 g. These miniature pieces were sterilized in a 70% alcohol/deionised water solution. After allowing the samples to dry for 2 hours, each sample was then soaked in 4 mL of Dulbecco's Modified Eagle's Medium (DMEM) tissue culture medium and placed in a sterilised 15 mL centrifuge tubes. These tubes were then placed in an incubator at 37° C. under 5% CO₂ atmosphere in order to simulate similar conditions that you would find in the human body. The acidity measurements were carried out by using a pH meter while the samples were still inside the incubator at the prescribed environmental conditions. On day 0, the first acidity measurement was made after incubation of the samples had preceded for 4 hours when the conditions in the incubator matched 37° C. and 5% CO₂ atmosphere. These measurements were repeated 24 and 48 hours later on day 1 and day 2 respectively to determine the pH levels over the testing period.

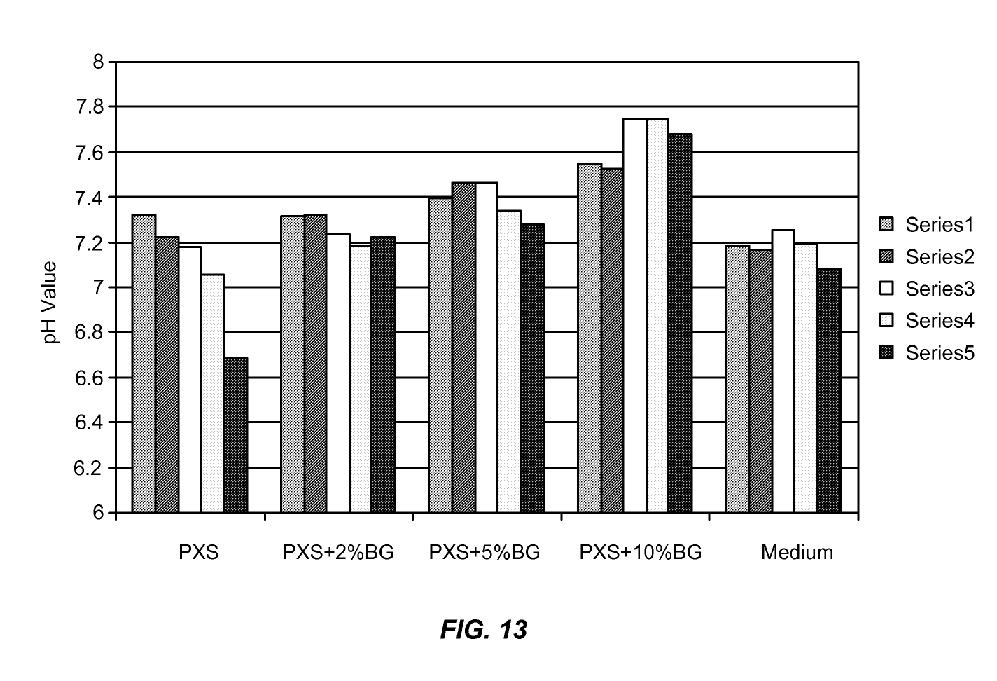

FIG. 13 illustrates the pH values of culture medium when incubated with PXS-BG composite samples. It was revealed that the pH value of the culture microenvironment could be maintained at the normal (nearly neutral) level of the body with 2, 5 and 10 wt % PXS-BG composites, and shows an improvement in pH stability compared to even for the PXS blank where there was a drop in the pH of almost 1 pH unit in series 5 after a period of time.

### EXAMPLE 12

### Mechanical Properties of PXS-BG Composites

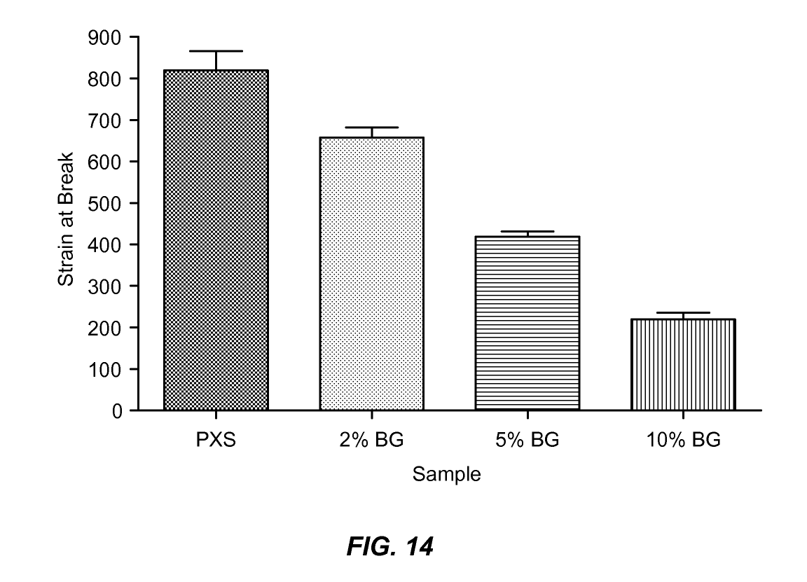

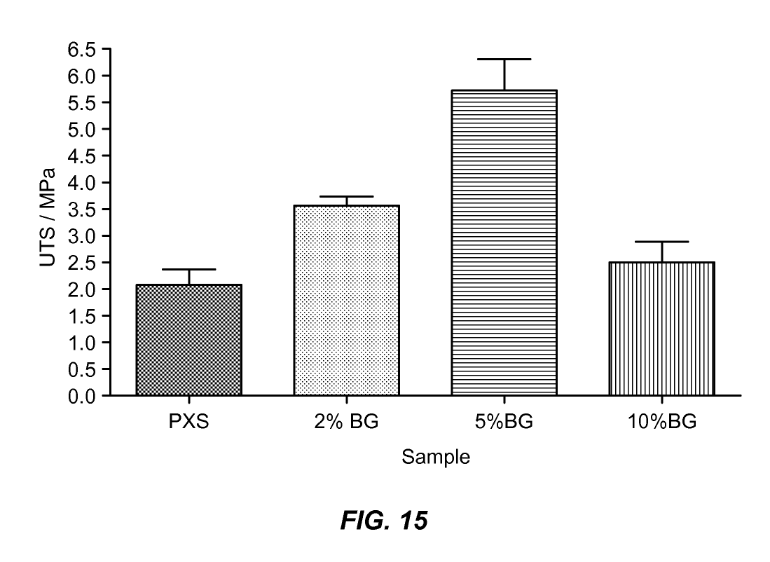

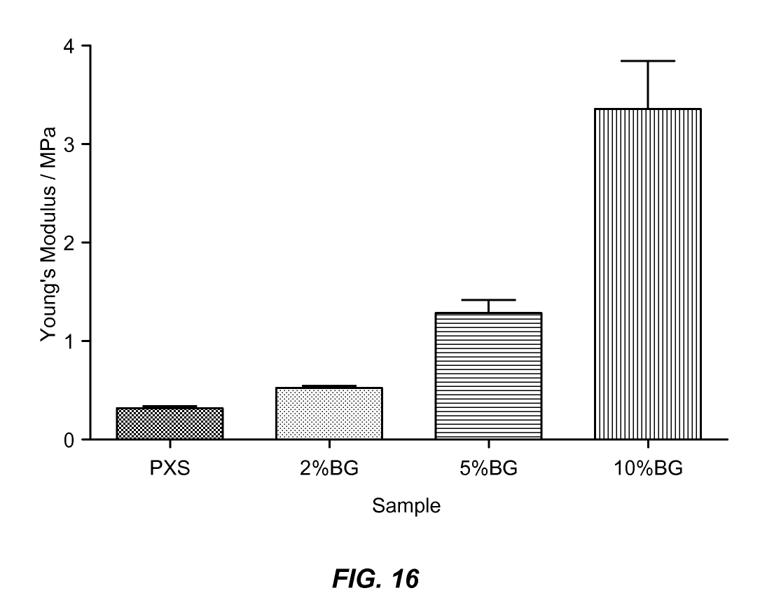

Mechanical properties for each of the PXS-BG composites were determined including elongation at rupture (FIG. 14), ultimate tensile strength (UTS, MPa) (FIG. 15) and Young's modulus (FIG. 16). The UTS and young's modulus increased with the percentage of added BG to the PXS polymer system reaching a maximum at 5% before decreasing again at 10%. The strain at rupture decreased first with the increasing of BG concentration.

### EXAMPLE 13

### Fabrication of poly(polyol) crosslinked polymer networks

Additional poly(polyol) polymer networks may be prepared by reaction of a polyol and other carboxylic acids, for example, citric acid, which contains three carboxylic acid groups as shown in Scheme 3.

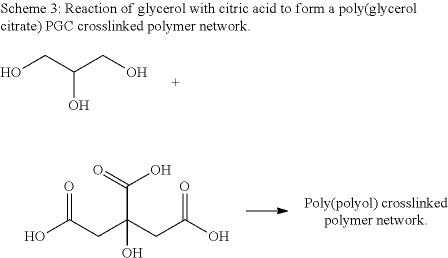

A polyol prepolymer may be synthesized by polycondensation of glycerol and citric acid at 110-150° C. under argon for 12-48 hr to produce a poly(glycerol citric acid) polymer (PGC). The prepolymer was then dissolved in a suitable solvent to produce a 50 wt/v % solution.

A series of PGC-bioceramic composites may be prepared by mixing BG powder into the PGC prepolymer solution prepared to produce 2, 5, 10 and 15 wt % percentage PGC-BG composites. As a reference, a PGC polymer may be prepared which contains 0% wt % BG. The slurries may then vigorously stirred for at least 1 hour and the resulting solution cast onto glass slides to produce sheet materials. The cast slurry may then be dried at ambient condition for 24 hours and under vacuum in oven for another 24 hours. Finally, the materials were then treated at 110-150° C. for 2-5 days to crosslink the PGC. The mechanical and degradation properties of the PGC-BG composite material can be manipulated by varying the degree of crosslinking (i.e. curing temperature, length of cure, amount of citric acid, etc).

### EXAMPLE 14

### Bone-Like Elastomer-Toughened Scaffolds with Degradability Kinetics Matching Healing Rates of Injured Bone

The replication technique used for fabrication of ceramic foams has been described elsewhere, see for example, Q. Z. Chen, I. D. Thompson, A. R. Boccaccini, Biomaterials 2006, 27, 2414. Briefly, 40 wt. % Bioglass® powder was added to a poly(vinyl alcohol) (PVA) water solution of concentration 5 g/100 mL, PVA being used as a binder. Polyurethane (PU) foam was soaked in the above glass slurry in order to coat Bioglass® particles onto the struts of polymer foam. The Bioglass®-coated PU foam was dried and sintered at 900-1100° C. for 1-3 hr, during which the PU foam was burnt out leaving glass-ceramic foam. In this investigation, the Bioglass®-ceramic foams were sintered at 950° C. for 1 h in order to achieve porous structure in the foam struts.

### EXAMPLE 15

### PGS Coating Procedures

The monomers of PGS were dissolved in THF at the ratio of 10 g PGS per 100 mL THF. Bioglass®-ceramic foams were soaked in the PGS-THF solution, during which the container was gently shaken so that the foams were coated homogeneously. After drying, the scaffolds were treated at 170° C. for 2 h. This step aimed at rapid polycondensation and to minimize the flowing of PGS by gravity, which would otherwise cause an inhomogeneous distribution of PGS in the scaffolds. The scaffolds were then treated at 120° C. for 2 or 3 days for crosslinking to occur.

### EXAMPLE 16

### Characterization Using EM, XRD and FTIR

The microstructure of the foams was characterized in a JEOL 7001 filed emission gun scanning electron microscope (FEG SEM), before and after immersion in simulated body fluid (SBF). Samples were gold-coated and observed at an accelerating voltage of 15 kV. Thin foils were prepared using the ultrathin sectioning technique, and examined by transmission electron microscope (TEM) JEOL 2011, at 200 kV.

Foams were also characterized using x-ray diffraction (XRD) analysis with the aim to assess the crystallinity after sintering and possible formation of HA crystals, after different times of immersion in a simulated body fluid (SBF). For XRD analysis, the foams were first ground into a powder. Then 0.1 g of the powder was collected. A Philips PW 1700 Series automated powder diffractometer was used, employing Cu K_{α} radiation (at 40 kV and 25 mA) with a secondary crystal monochromator. Data were collected over the range 2θ=5-80° using a step size of 0.02° and a counting time of 10 s per step. The measurement of Fourier transform infrared (FTIR) was performed on a Nicolet 6700 spectrometer. The spectrum was recorded with a resolution of 4 cm⁻¹.

Mechanical testing: The compression strength of foams was measured using an Instron Microtester 5848. The samples were rectangular in shape, with dimensions: 10 mm in height and 5 mm×5 mm in cross-section. During compression testing, the load was applied until densification of the porous samples started to occur.

Assessment of bioactivity in simulated body fluid: The bone bonding capability of a biomaterial to host bone is associated with the formation of a carbonated HA layer on the surface of the material when implanted or in contact with biological fluids. Hence, the ability to bond with bone can be assessed in vitro in simulated body fluid via monitoring the formation of HA on its surface, which was tested according to a method by Kokubo T, Hata K, Nakamura T, Yamamura T. in the article entitled “Apatite formation on ceramics, metals, and polymers induced by a CaO—SiO₂-Based glass in simulated body fluid”. In: Bonfield W, Hastings G W, Tanner K E, editors. Bioceramics 4. London: Guildford, Butterworth-Heinemainn; 1991. p. 113-20. The foams were immersed in 75 ml of acellular SBF in flasks. The flasks were placed inside an incubator at 37° C. The pH of the solution was maintained constant at 7.25. The size of all samples for these tests was 10 mm×10 mm×10 mm. Two samples were extracted from the SBF solution after given times of 3, 7, 14, 30 and 60 days. The SBF was replaced twice a week because the cation concentration decreased during the course of the experiments, as a result of the changes in the chemistry of the samples. Once removed from the incubation, the samples were rinsed gently, firstly in pure ethanol, then using deionised water, and finally left to dry at ambient temperature in a desiccator.

Biocompatibility evaluation: Elution test method: Mouse fibroblasts, SNL (STO-Neo-LIF) (SNL), were used for the initial assessment because of their defined and reproducible proliferative activity. Elution test method (ISO 10993) was adopted in the present work. In this method, extracts were obtained by placing the test (Bioglass®-PGS composite) and control (PDLLA) materials in separate cell culture media under standard conditions (0.2 g/ml of culture medium for 24 h at 37° C.). SNL cells were cultured in DMEM with 10% heat-inactivated foetal bovine serum, 0.1% penicillin/streptomycin at 37° C. with 5% CO₂. Cells were then plated on a 48-well tissue culture plate at a concentration of 2×10⁴ cells/well. After 2-day culture, cell culture media was removed and replaced with the media containing the extractants. Cells were placed back in the incubator for a 24-h treatment. Cells are observed for visible signs of toxicity in response to the test and control materials.

Quantization of cell viability was achieved by measuring lactate dehydrogenase (LDH) release, using a commercial kit (Sigma-Aldrich Tox-7). Culture media (200 μm per well) were collected after above SNL cells exposed to the media containing extracts. The number of dead cells during the treatment by extractants was determined from these samples. The number of live cells was measured using the total LDH method of Tox-7, in which live cells were lysed and the media were collected. The LDH levels were determined by measuring the absorbance (A₄₉₀-A₆₉₀), using the commercial kit Tox-7 and spectrophotometer. Our standard curve (appendix A) shows that there is a reasonably good linear relationship between the number of cells and LDH level in the range of 5×10³-5×10⁴. Hence, the percentage of dead cells can be expressed by

$$\frac{LDH of extractant medium}{Total LDH} \qquad ( 1 )$$

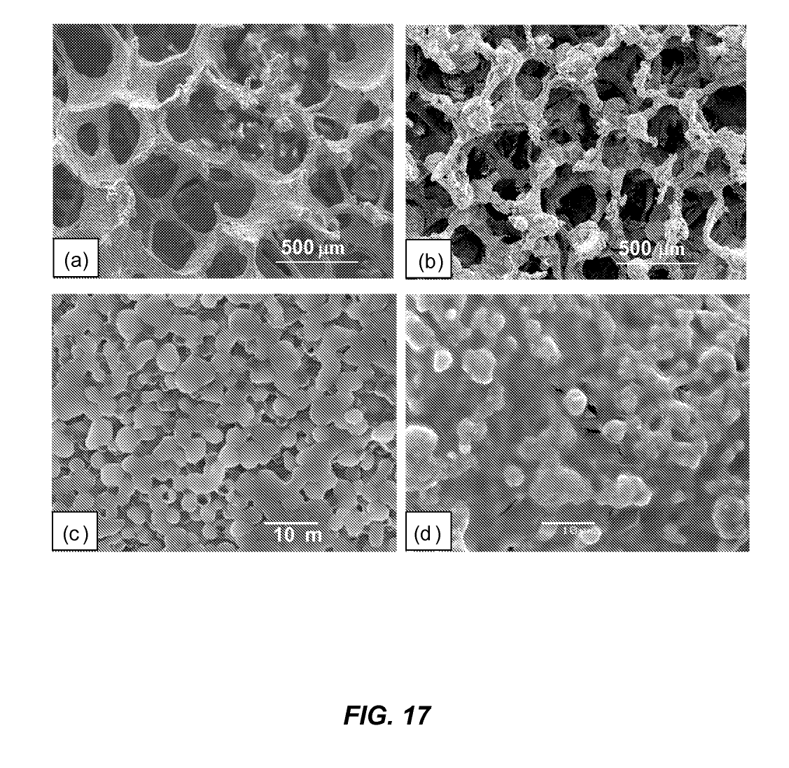

Improved mechanical properties of as fabricated scaffolds: FIG. 17 shows the porous network and microstructure of the foam struts before and after coating of PGS. The highly porous and connective network was maintained after the coating (FIG. 17a-b), and microvoids on the foam struts (FIG. 17c) were filled with PGS (FIG. 17d). The cracks in the coating layer of PGS in FIG. 17d were induced by the electron radiation during examination.

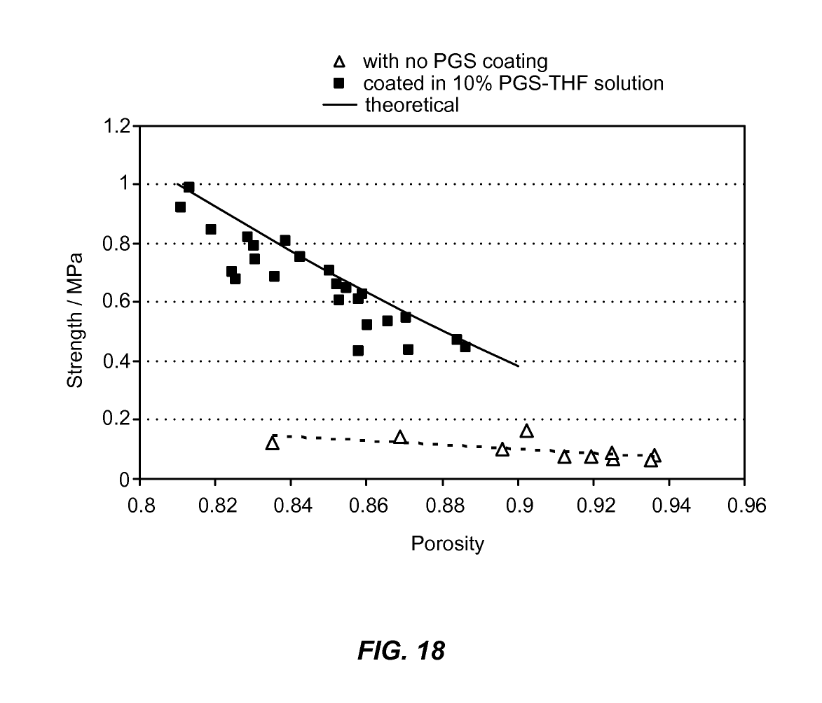

Compressive mechanical strengths of PGS-coated scaffolds were significantly improved, compared with uncoated foams. FIG. 18 shows the compressive mechanical strength values of the two groups of foams. The theoretical strength values (the solid line), which were calculated using Gibson and Ashby's theory, represent the upper bound of the strength of porous scaffolds. It can be seen from FIG. 18 that the crosslinked PGS coating, which reduced the porosity about 0.05 on average, pushed the strength of the scaffolds toward the upper limit of the strength values of porous networks. Theoretically, no experimental strength value could go beyond the upper bound. Hence, the two points that are above the theoretical strength line in FIG. 18 could be attributed to the experimental errors. One of error sources could be the size measurement of the highly porous foams.

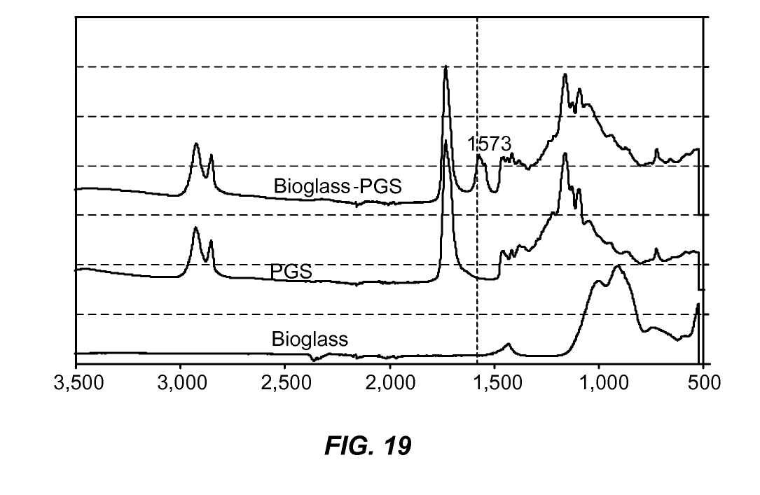

Strengthening mechanism in as fabricated scaffolds: In the present work, the PGS coating, which infiltrated into the microstructure of the foam struts, was treated at 120° C. for two days for crosslink. During the crosslink treatment, an acid-base reaction was expected to occur at the interface of the acidic PGS and alkaline Bioglass®-ceramic due to partially dissolving of the particles. The expected chemical reaction was confirmed by the FTIR analysis, as shown in FIG. 19. A new peak appears at the frequency of 1573 cm⁻¹ in the spectrum of Bioglass®-PGS. This peak is attributable to the metallic carboxylate groups, in particular —COONa. In 45S5 Bioglass® (SiO₂—Na₂O—CaO—P₂O₅), sodium oxide is the most active component. Indeed, Na₂O has been used in glass industry to reduce the melting point of silica-based glasses, whereas other components (e.g. CaO) are added to stabilize glass. It has previously been shown that the release of sodium ions from Bioglass®-ceramic took place immediately after soaking in water. Hence, the carboxylic acid group —COOH could largely be carboxylated by Na⁺.

Without wishing to be bound by theory, it is thought that the strengthening is the result of bonding between the PGS and BG components of the composite. The chemical reaction between Bioglass®-ceramic and PGS was metallic carboxylation. This chemical reaction formed a fusion, bonding layer around each Bioglass®-ceramic particles. As a result of the strong chemical bonding between PGS and Bioglass®-ceramic particles, the mechanical strength of the composite scaffolds was greatly improved towards the upper limit.

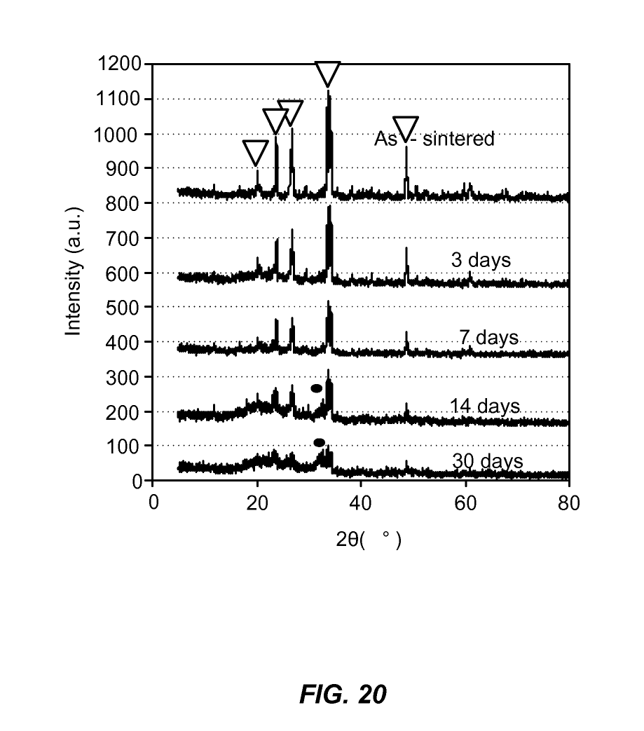

Stable mechanical performance during degradation in vitro: During the first month of soaking, however, the PGS-Bioglass® material did show clear signs of degradation of its original crystalline structure at the microscopic level, as indicated by XRD analysis (FIG. 20). The diffraction peaks of crystalline phase Na₂Ca₂Si₃O₉ formed during the sintering of Bioglass® foam became shorter with increasing incubation time in aqueous medium, eventually disappearing after incubation for 30 days and leaving a broad halo pattern (characteristic of amorphous structure) overlapped with weak apatite peaks. The formation of apatite also indicated a good bone-bonding ability of the new composite scaffolds. If heat treated at 120° C. for 2 days, the composite scaffolds maintained a mechanically steady state for up to 2 weeks, with significant decrease in compressive strength manifested only after the samples were soaked for 30 days, indicating that the duration of the steady state can be tuned purely by modifying the synthesis conditions of the composite foams.

It was found that the coating of PGS neither slow down the structural degradation of Bioglass®-ceramic substance nor impair the bone-bonding ability of Bioglass®-ceramic, as indicated in FIG. 20. For both PGS-coated and uncoated scaffolds, the diffraction peaks of crystalline ceramic phase, Na₂Ca₂Si₃O₉, became short with increasing of incubation time in SBF, eventually disappeared after incubation for 30 days, leaving a broad diffraction hill (indicting amorphous) overlapped with weak apatite peaks.

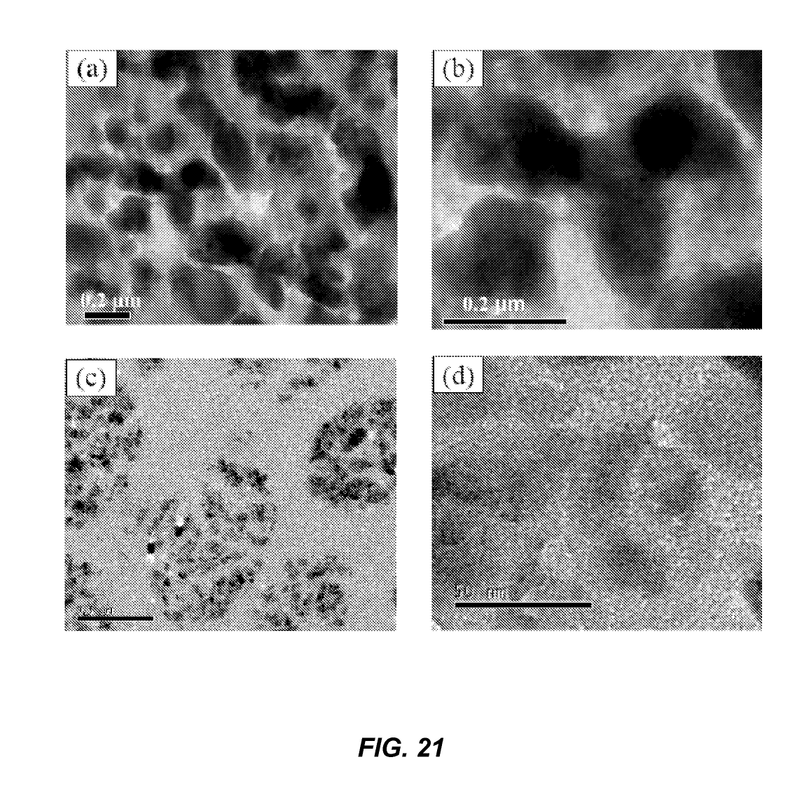

Transmission electron microscope (TEM) examination was carried out on the PGS-Bioglass® samples heat treated at 120° C. for 3 days and soaked in tissue culture medium for 1, 3, 7, 14 and 30 days. The analysis revealed that the surface dissolution of Bioglass®-ceramic particles was the main character at day 3, as shown in FIG. 21(a-b). Fine precipitates (˜50 nm in size) were evident after incubation for 14 days and 30 days, as shown in FIG. 21(c). This morphology indicates that a cluster of nanoparticles was derived from one original micro-sized particle. Furthermore, the nanoparticles were embedded and fused with the polymer matrix at their interfaces (FIG. 21b). Little evidence showed that the formation mechanism of these nanoparticles was just breaking up of large particles into small particles, as this mechanism would have resulted in gaps between fine particles. Rather, the morphologies in FIG. 21 indicate a dissolution-reprecipitation mechanism that was reported for in-vivo degradation of Bioglass® implants, i.e., dissolution of large Bioglass® particles into the surrounding matrix and formation of an inorganic-organic gel, which is followed by precipitation of apatite nanoparticles from the gel. Hence, we conclude that the mechanical steady state of the composite scaffolds during the early period of degradation is a result of the strengthening effect of nanosized apatite particles that are precipitated from the dissolution products of the Bioglass®.

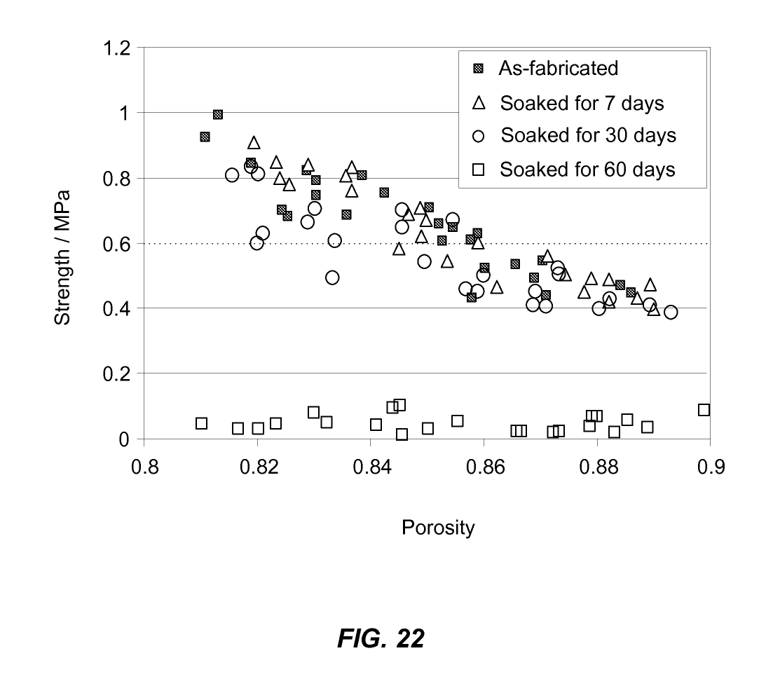

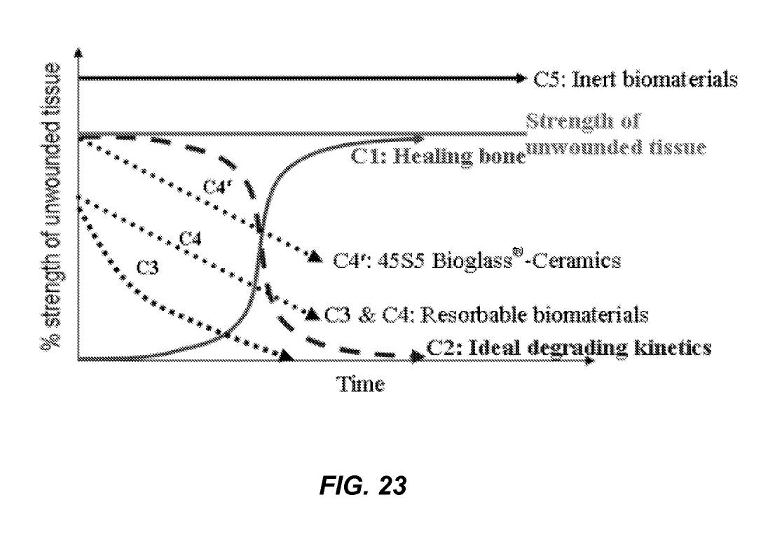

However, it was surprisingly discovered that the mechanical strength values of the Bioglass®(ceramic)-PGS composite scaffolds remained at the same level up to 30 days (FIG. 22) while the Bioglass-ceramic was degrading microscopically in SBF. This unexpected mechanical performance is of great importance to achieve a mechanically steady state of bone implants at the initial period of post implantation. The time course of healing tissue exhibits three stages: lag, log and plateau phases, as illustrated in FIG. 23 (curve C1). Accordingly, ideal degradation kinetics of scaffolds that match the healing rate of growing bone should possess three stages as well, i.e. lag (a steady state), log (rapid degradation) and plateau (end of degradation) phases (FIG. 23, C2). Unfortunately, current biomaterials either degrade immediately after implantation, showing no lag phase (FIG. 23, C3 or C4), as seen with many degradable biomaterials that are weaker than mature (cancellous) bone, or they are virtually inert and degrade poorly (FIG. 23, C5), which is typical of more mechanically robust biomaterials. Hence, a highly desirable scaffold is expected to be able to maintain mechanical strength during the initial lag growth period of host bone tissue post implantation, and only start to degrade when the growth of new bone tissue enters the log phase. In reality, however, this criterion seems difficult because all existing degradable implants would mechanically deteriorate immediately from the moment of implantation due to the structural breakdown of the degradable biomaterials, as demonstrated in FIG. 23. This is compared to the results shown in FIG. 22, from which the ideal degradation kinetics (inset in FIG. 22) desired by bone tissue engineering may be achievable.

Biocompatibility of the composite scaffolds: In order to determine the potential clinical usefulness of the PGS-Bioglass composite, it was necessary to undertake in vitro biocompatibility assessments on the material. Osteoblast-like (MG63) cells were used for the preliminary assessment, employing the elution test method (ISO 10993). Quantitative assessment of cell viability and proliferation showed no differences between the current PGS-Bioglass® material, the tissue culture plate (GMP plasma-treated polystyrene) and poly(D,L-lactic acid) (PDLLA), indicating similar biocompatibility to accepted biocompatible polymers used in vitro and clinically.

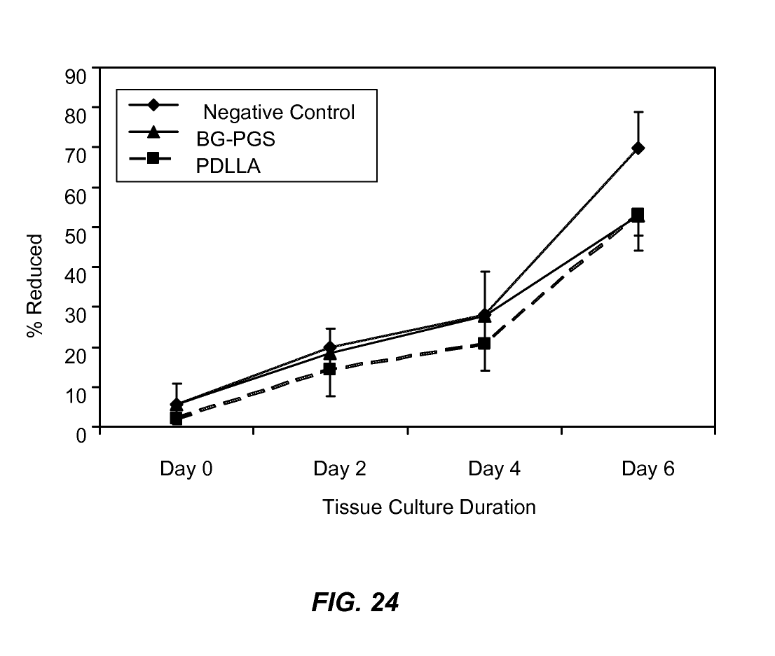

It was found that SNL cells proliferated equally well in the three culture media: normal culture medium, medium with PDLLA or Bioglass®(ceramic)-PGS extracts. There were no significant differences in the percentage of dead cells (FIG. 24). Hence, the newly developed Bioglass®(ceramic)-PGS composite is satisfactorily safe in terms of cytotoxicity, being comparable to the clinically applied polymer PDLLA.

In conclusion, these composite scaffolds have very similar mechanical strength to that of cancellous bone of the same porosity, and exhibit a mechanically steady state over an extended period in a physiological environment, while undergoing controlled microstructural degradation. The second feature is of great importance to bone tissue engineering, where a lag phase of degradation following implantation is highly desirable, in order to provide support to the damaged or fragmented bone. A subsequent, rapid degradation could allow for the recovering bone to infiltrate and replace the implant. This work shows that the ideal degradation kinetics in mechanical function that matches the healing process of host bone (C2 in FIG. 23) is achievable with the present synthetic composite under physiological conditions.

The Bioglass®(ceramic)-PGS composite scaffold has unique mechanical properties that have not been reported with currently existing scaffolds. First, it possesses a predictable mechanical strength that is close to theoretical strength value. Second, it has a mechanical steady state over a period when immersed in a physiological environment while the two components of the composite are structurally biodegrading. Moreover, the composite system has a bone-bonding ability, as well as an excellent biocompatibility.

### EXAMPLE 17

### Elastomeric Nanocomposites as Cell Delivery Vehicles and Cardiac Support Devices

Equivalent amounts of calcium and sodium 2-ethylhexanoate were mixed with hexamethyldisiloxane and tributylphosphate and diluted with xylene. The solution was pumped (10 ml min−1) through a capillary (diameter 0.4 mm), dispersed with oxygen (10 l min−1) and ignited with a methane (1.13 l min−1) and oxygen (2.4 l min−1) flame. The as-formed bioactive glass particles were collected by using a baghouse filter and they were then sieved with a 250 μm mesh sieve to separate the agglomerates.

A PGS prepolymer was synthesized by partially condensing the water byproduct from an equimolar mixture of glycerol and sebacic acid at 120° C. under nitrogen for 24. The nanocomposites were fabricated by blending nanoparticles of Bioglass® into the PGS prepolymer prior to its cross-linking. The Bioglass® powder was mixed into the prepolymer at 50° C. at concentrations of 2, 5 and 10 wt. %. This was followed by casting of the above mixture on glass slides to prepare sheets of the composite. Finally the cast mixture was cured at the same temperature under vacuum conditions for either 2 or 3 days—since the formation of the elastomers is by loss of water during esterification, the longer crosslinking period was expected to increase the crosslink density of the elastomer. After cooling to room temperature under vacuum, the 0.2-0.3 mm thick sheets of PGS-Bioglass® composites were peeled off the glass slides.

Samples of the thus prepared PGS-Bioglass® were examined for acidity, mechanical tensile strength, Fourier transform infrared spectroscopy (FTIR), swelling test, cytotoxicity, cell proliferation and hESC-dervied cardiomyocytes.

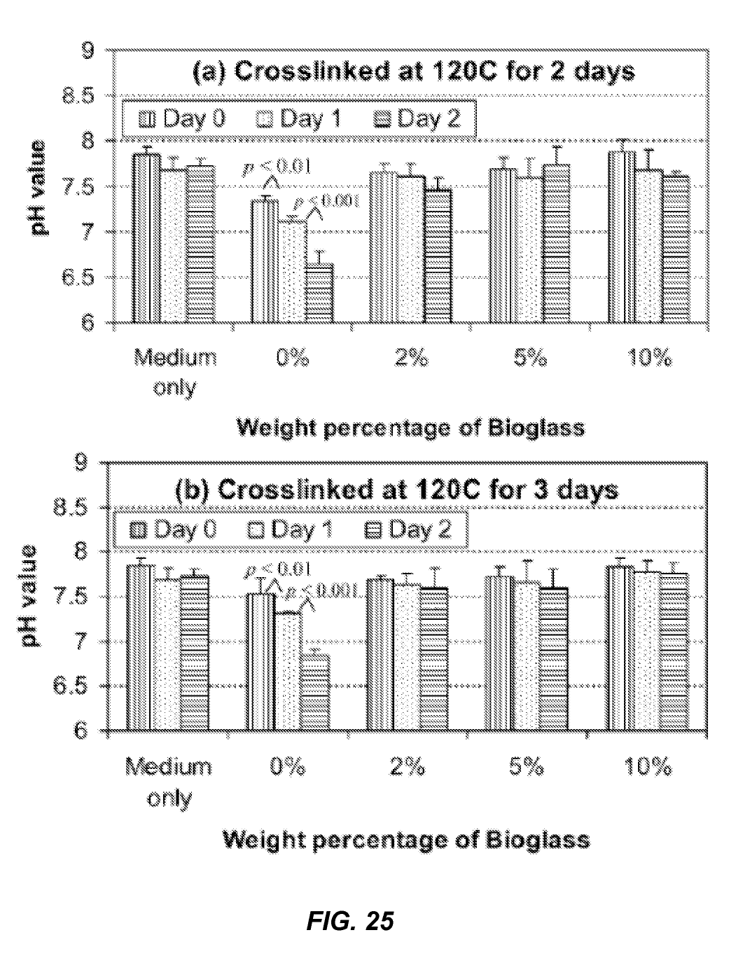

Acidity of tissue culture medium: The effect on the pH of culture medium by the presence of either of the two pure PGS materials (crosslinked at 120° C. for either 2 or 3 days) was studied and it was observed that the acidity level of culture medium increased significantly (p<0.01) after soaking of the PGS specimens (FIG. 25). After two days of soaking, the culture medium was more acidic (pH≈6.6 on average) when in contact with the PGS specimen cured at 120° C. for 2 days than for the specimen cured at 120° C. for 3 days (pH≈6.8 on average). This can be attributed to the higher crosslink density of PGS when polymerized for a longer period. The effects of crosslink density on acidity are two-fold: firstly, a higher crosslink density reduces the number of unreacted carboxylic acid groups and so reduces acidity; secondly a higher crosslink density also slows down water diffusion into the chain network and thus reducing the hydrolysis (i.e. cleavage of ester bonds) kinetics.

The presence of alkaline Bioglass® in the nanocomposites of all three compositions effectively counteracted the acidity caused by the degradation of PGS, as indicated in FIG. 25. No significant reduction in pH value occurred to the media that were incubated with any of the Bioglass®-filled nanocomposites (p>0.05).

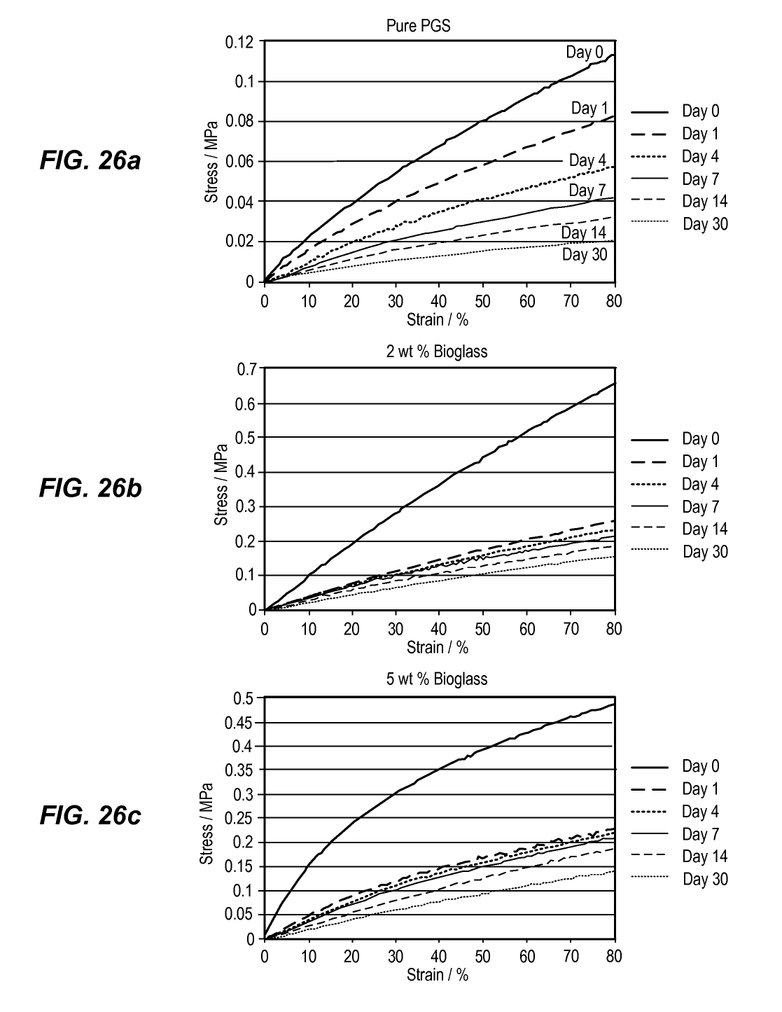

Mechanical properties of PGS and its nanocomposites: FIG. 26 illustrates the stress-strain curves of the polymers containing 0, 2, 5 or 10 wt % Bioglass® which had been crosslinked at 120° C. for 2 days and incubated in culture medium under standard culture conditions. The slope of the stress-strain curve of the unfilled PGS sample dropped slowly over time as shown in FIG. 3a. In contrast, the stress-strain curves of the nanocomposite samples experienced a sudden drop after one-day incubation and then dropped more slowly over time (see FIG. 26b-c for 2 and 5 wt % filled samples; composites of 10 wt % showed similar profiles). The above phenomena were also observed in the materials crosslinked at 120° C. for 3 days, not shown. However, here the sudden drop in slope of the stress-strain curves was only observed in the nanocomposites with a filler level of 5 wt % or 10 wt %, but not in the composite with 2 wt % Bioglass®.

The strain in the heart wall of a normal heart is typically 15% at the end of diastole. Hence, the stress in the heart patch and thus its modulus at small strains (<15%) is relevant to the clinical application scenario.

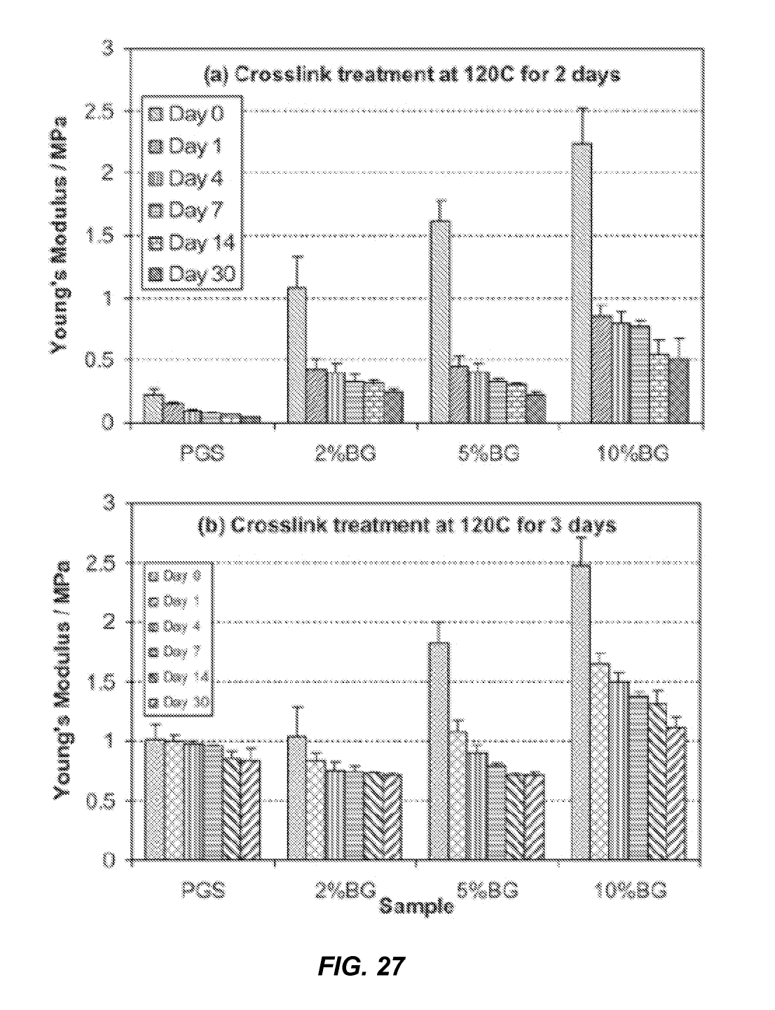

FIG. 27 illustrates the small-strain Young's moduli of PGS and nanocomposites before and after incubation in culture medium. Pure PGS polymers treated at 120° C. for 2 days are very soft, with Young's modulus being ca. 0.22 MPa (FIG. 5a). The addition of nanoBioglass® greatly stiffened the material, with the Young's modulus increasing by 5, 8 and 10 times in nanocomposites with 2, 5 and 10 wt % Bioglass®, respectively, as shown in FIG. 27a. However, the rigidity of the composites caused by the addition of the nanoBioglass® filler rapidly dropped after one day incubation, followed by a more gradual reduction in Young's modulus. The Young's moduli of composites with 2 and 5 wt % Bioglass® were already below 0.5 MPa after only one-day soaking, which are within the range of the desired stiffnesses of heart patches.

Unfilled PGS crosslinked at 120° C. for 3 days was relatively stiff, with a Young's modulus of ˜1 MPa, and similarly, the polymer's rigidity rose rapidly with addition of nanoBioglass®(FIG. 27b). When cured at 120° C. for 3 days, the stress-strain curves of pure PGS and of nanocomposite with 2 wt % filler both dropped slowly in tissue culture medium due to low permeability. However, a rapid drop in Young's modulus occurred with nanocomposites of 5 and 10 wt % Bioglass® after soaking in culture medium. Unlike the materials crosslinked at 120° C. for 2 days, the Young's modulus of the materials treated at 120° C. for 3 days generally remained higher than 0.5 MPa after soaked in culture medium. Hence, the materials that were crosslink-treated at 120° C. for 3 days could likely be too rigid to be used as a heart patch in the present application strategy.

The strains at rupture of the present materials are much larger than the maximal strain (12-15%) of heart muscle in vivo and hence are suitable on this basis for the application. In addition, the strains at rupture directly indicate the strengthening mechanisms of the Bioglass® fillers in the elastomeric matrix. In general, the strains at break were initially increased with the addition of the nanoparticles. For instance, in the materials crosslinked at 120° C. for 3 days, the maximal strain increased from ˜100% in pure PGS to more than 200% in 5wt % filled-composite. However, the strain at rupture began to decrease with further increase of nanoBioglass®. The maximal strain in the composites of 10 wt % filler, for example, was smaller than those of 5 wt % filler. This reduction was probably caused by the poor quality of these composites because it was very difficult to produce a homogenous and defect-free nanocomposite (e.g. without microvoids, and microcracks) with a high percentage of filler.

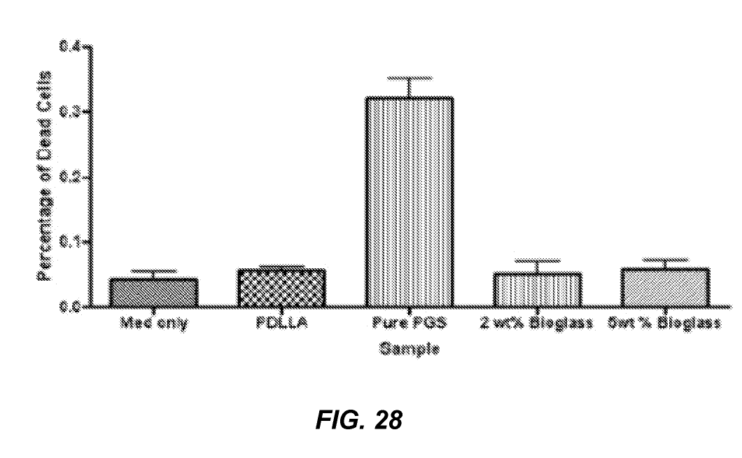

Cytocompatibility-mouse fibroblasts: The evaluation of biocompatibility was conducted on the most promising materials, i.e. the materials that were crosslinked at 120° C. for 2 days. Visual observation found that cells remained normal after one-day culture in the extractant media of all the materials. However, cellular toxicity was manifested in the cultures containing extracts of pure PGS, while the media containing the extracts of nanocomposites were found to support proliferation of SNL cells. Quantitative assessment using LDH technique confirmed that the proportions of dead cells were significantly lower in SNL cultures exposed to the extracts of nanocomposites (p<0.01) than those to the extracts of the pure PGS (FIG. 28). Further more, the growth kinetics of SNL cells were significantly higher in the media containing composite extracts than in those containing the extracts of the pure PGS (p<0.01).

Cytocompatibility: hESC-derived cardiomyocytes: To assay the effects of extractant media on cardiomyocyte viability, human ESC-derived embryoid bodies containing contractile cardiomyocytes (hESC-CM) were cultured in extractant media of the three PGS-nanoBioglass® composites from day 14 of differentiation. No significant difference was observed between hESC-CMs cultured in standard medium (BEL) and hESC-CMs cultured in extractant media (FIG. 13). Values of beating rates for hESC-CM in extractant media at various time points lie within the range for hESC-CM in the standard culture medium (their ideal environment) indicating that the nanocomposites do not inhibit functional activity of hESC-CM.

The nanocomposites have been characterised in terms of materials science and evaluated for their potential clinical application as cell delivery vehicles and cardiac support devices in the heart patch strategy. The addition of alkaline Bioglass® effectively counteracts the acidity caused by the degradation of PGS without severely compromising the compliance of PGS. As a result, the newly developed PGS-nanoBioglass (<5 wt %) composites have a greatly improved biocompatibility, compared to PGS, while and remains mechanically compatible to the with heart muscle. The interaction between PGS and Bioglass® and reinforcement of the PGS polymer network by the nanoBioglass® particles have also been explored in depth.

### EXAMPLE 18

### Manipulation of the Degradation and Compliance of Elastomeric PGS by Incorporation of Halloysite Nanotubes for Soft Tissue Engineering Applications

All precursors of the materials were purchased from Sigma-Aldrich. The average tube diameter and inner lumen diameter of the halloysite are ˜100 and 85 nm, respectively. The typical specific surface area of the halloysite is ˜65 m²/g; with pore volume being ˜1.25 mL/g, refractive index being ˜1.54 and specific gravity being ˜2.53 g/cm³. The crosslinked PGS and composites were prepared in two stages. Initially a PGS prepolymer was synthesized by polycondensation of 1:1 molar ratio of the triol, glycerol (purity 99%) and the diacid, sebacic acid (purity 99%). Note in this formulation the ratio of carboxylic acid groups to alcohol is 2:3; thus at a 100% conversion of the carboxylic groups, excess alcohol groups (33%) remain. The polycondensation reaction was initially carried out at 120° C. for 24 hours under nitrogen gas—at this stage, the reaction was incomplete and the prepolymer was still ungelled and could be melted at 50° C. The molecular weight of the PGS prepolymer was determined by gel permeation chromatography (GPC) using THF on PLgel columns (10 μm, 1000 A, Mw 1 k-40 k). Six percentages (0, 1, 3 5, 10 and 20 wt. %) of halloysite were added to a melted PGS pre-polymer at 50° C. and magnetically stirred thoroughly. The slurry was then cast onto glass slides and cooled at ambient conditions to produce ˜0.5 mm thick pre-sheets of PGS or PGS/halloysite composites. Finally, the cast sheets were polymerized under vacuum at 120° C. for further 3 days to increase the crosslink density of the final material. The resultant PGS/halloysite composites were analysed using microanalysis, FTIR and TEM.

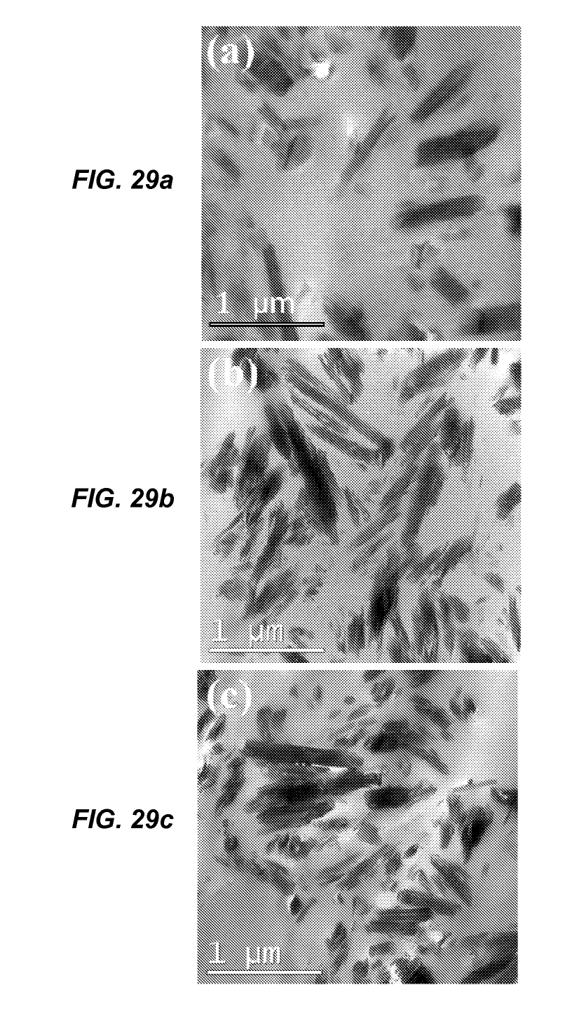

TEM observations on the PGS/halloysite composites (FIG. 29) revealed that the halloysite nanotubes were uniformly distributed in the PGS matrix when the filler composition was 1-20 wt %. FIG. 29a-b shows the typical morphology in the composite of 3-5 wt % halloysite, and no agglomeration was observed in all examined TEM foils of 3-5% PGS/halloysite materials. Whilst halloysite nanotubes were distributed uniformly at most areas in the 10 and 20 wt % composites (FIG. 29c for 10 wt %), agglomeration was occasionally observed. This confirms that the method used to synthesise PGS/halloysite composites was reliable and ensured that measurements of the properties of the materials were reproducible with small standard deviations, especially at low filler levels (≦5 wt. %).

Acidity measurement of halloysite in deionised water: This study aimed to understand the effect of halloysite on the crosslinking kinetics of PGS in composites. Halloysite at concentrations of 0, 1, 3, 5, 10 and 20 wt % were added into deionised water in 50-ml corn tubes. The tubes were placed in a shaker for 24 hrs prior to pH measurement. Acidity was measured using an electrode (Hanna® Instruments, HI 1230B) attached to a pH meter (Hanna® Instruments, HI 98185).

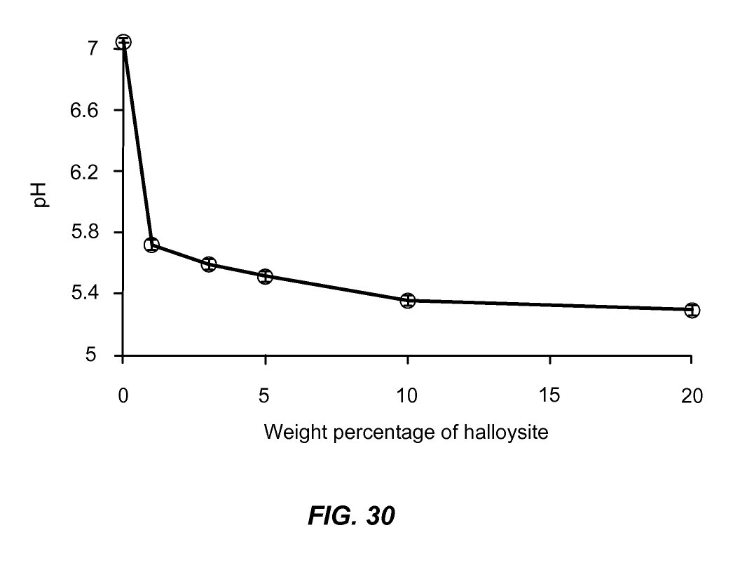

Compared with the control samples (i.e. deionised water) which had not been in contact with the halloysite, the pH values of the water in contact with the halloysite at the five weight percentages were all significantly lower (p<0.001), indicating that acidification had taken place (FIG. 30). The reduction in pH increased with the increment of halloysite component, reaching a saturated value around 10 wt % (note: the pH value in the 20 wt % slurry was not significantly lower than that of the 10 wt % slurry, p>0.05).

Mechanical properties of PGS and PGS/halloysite composites: Dog-bone shaped specimens of 12.5×3.25×t mm (length×width×thickness) were cut for testing. Tensile and cyclic tests were performed at room temperature with an Instron 5860 mechanical tester equipped with an 100N load cell, and at a cross-head speed of 10 and 25 mm/min respectively, according to previous work. For studies of the virgin materials, the specimens were stretched to failure. For experiments of the effect of culture medium on mechanical properties, the specimens were stretched to a strain level of 50% (well below the breaking strain) so that the same specimens could be reintroduced into the culture medium (with no mechanical loading) and re-tested at different intervals of the degradation process. The cyclic test specimens were stretched at the rate of 25 mm/min according to previous work. Since the maximum strain of dynamic loading required of soft tissues, such as cardiac muscle, is typically around 15% in normal physiological conditions, the cyclic test specimens were stretched to a strain of 15%.

For the same reason, the mechanical behaviour at low strains (<15%) is relevant to clinical applications. Since the polymer and composites investigated here were in the rubbery state, their stress-strain behavior can be described by the equation of rubber elasticity. At low strains, this equation relating stress (σ) to strain (ε) or extension ratio (λ) can be linearized with an error of 8.8% when ε=10%. Hence, the Young's modulus of each specimen was determined by σ/ε at a strain of 10%. Resilience was calculated from the ratio of the area under the relaxation curve to the area of under the extension curve at the strain of 15%.

Static mechanical properties—Tensile: PGS and its halloysite composites showed stress-strain curves which are typical of elastomers at room temperature. As is consistent with the deformation behaviour of an elastomer, no stress whitening or plastic deformation were visually observed in the samples during the tensile tests.

The average values of Young's modulus (E), ultimate tensile strength (UTS) and strain at break (εₘₐₓ) were all observed to increase in the composite, slightly at low concentrations of halloysite and more significantly at 10 and 20 wt % of halloysite. E increased nearly two-fold (0.80±0.10 to 1.51±0.04 MPa). The UTS increased more than two-fold (0.60±0.06 to 1.60±0.16 MPa), whilst εₘₐₓ increased from 110±22% to 225±10% with the addition of halloysite. However, the strain at rupture showed a greater degree of data scatter for further halloysite additions, which could be attributed to the agglomeration of halloysite for high percentage halloysite additions. Thus, in these PGS nanocomposites, the addition of nanotubular halloysite did not compromise the extensibility of material, compared with the pure PGS counterpart. Instead the elongation at rupture was increased to 225% (indicating good interaction between polymer and nanofiller), whilst the Young's modulus of 1-5 wt % composite remained close to the level of pure PGS. Hence the increase in UTS of these composites was due to the large strain at rupture, rather than by a significant change in the Young's modulus.

Dynamic mechanical properties—Tensile/cyclic: The cyclic stress-strain curves of PGS and its halloysite composites indicate that the mechanical properties of the present materials were very stable, varying slightly during cyclic testing due to a stress softening effect. The resilience was on average 96, 96, 98, 94, 91 and 90% in PGS and 1, 3, 5, 10 and 20 wt % PGS/halloysite composites, respectively, all being greater than the resilience of biological tissues (90%), including collagen and elastin. The overall drop in stiffness after the 10 cycles was, on average, 1.0, 1.6, 1.5, 1.3, 5.6 and 9.5% in PGS and 1, 3, 5, 10 and 20 wt % PGS/halloysite composites. A comparison of the data reveals that the Young's moduli varied little with the strain rate, which was 10 and 25 mm/min in tensile and cyclic testing, respectively.

The addition of halloysite slightly increased hysteresis in the composites, which was reflected by the drop in resilience from 96% of pure PGS to ˜90% in 10 and 20 wt % PGS/hallosite composites. An additional mechanism increasing the hysteresis in the present composites was the reduction in the level of esterification crosslinks in the PGS matrix of these materials, as it has been found that crosslinking leads to an increase (decrease) in the elasticity (hysteresis) of the rubbers.

Prolonged degradation of mechanical properties in vitro: Tensile test specimens were incubated in Dulbecco's Modified Eagle Medium (DMEM, GIBCO® 11965) culture medium in a culture incubator at 37° C., under 5% CO₂ for up to one months. The medium was changed every second day. Each specimen was taken out at different time intervals (1, 3, 7, 14, and 30 days) and tested in the tensile testing machine to a strain level of ˜50% (which is well below the breaking strain). After unloading, the specimen was placed back in medium and incubated until the next tensile testing.

The stress-strain curves of the present PGS and PGS/halloysite materials all declined (bended downward) with the prolonging of incubation time. Pure PGS and 1 wt % composite had similar profiles, with stress-strain curves, declining gradually and steadily with time. PGS nanocomposites of 3-5 wt % halloysite content exhibited relatively stable stress-strain curves following prolonged incubation in culture medium of up to 30 days, with Young's modulus decreasing slightly. In contrast, for the nanocomposites of 10 and 20 wt %, the stress-strain curves declined rapidly.

The dependence of Young's moduli on the immersion time in culture medium also revealed marked differences between PGS composites of 3-5 wt % and the other samples. As expected, the modulus decreased steadily with PGS (from 0.8 to 0.4 MPa) and rapidly with 10 wt % composite (from 1.2 to 0.3 MPa); whereas 3 and 5 wt % composites showed a slow degradation in Young's modulus over the 30-day incubation period.

The degradation rate was influenced by two opposite factors in the PGS/halloysite composition: first, the crosslink density of PGS network was reduced in the composite due to the acidic effect of halloysite nanotubes; and second, in the bound rubber layer the densely absorbed macromolecules could effectively hinder the water attack and thus reduce the hydrolysis rate. It was possible that the bound rubber effect outweighed the acidic effect in the composites of 3 and 5 wt %, resulting in a reduced degradation rate in these materials. If the bound rubber effect was overwhelmed by the acidic effect, the rate of hydrolysis may be accelerated, and this may be the case for the composite of 10 and 20 wt %.

These results indicate that the addition of halloysite filler could be a control of degradation kinetics, which is independent of their mechanical properties of the materials; and thus offer an opportunity to achieve a satisfactory balance of degradation rate and flexibility simultaneously in an elastomeric material.

The cyclic stress-strain loops of PGS and its halloysite composites remained reasonable narrow in 0-5 wt % materials after soaking in DME for one month, which indicate that the elasticity of these materials was not significantly deteriorated. This conclusion was also supported by the resilience data, which decreased slightly over the incubation time. However, large hysteresis occurred in the composites of 10 and 20 wt %, especially after one-month incubation.

The overall mechanical and degradation performance indicates that the composites of 3 and 5 wt % are the most promising ones, with a nearly unchanged compliance compared with the pure PGS counterpart, significantly reduced degradation rates, and well maintained elasticity.

Biocompatibility of PGS/halloysite nanocomposites: SNL mouse fibroblasts were used to conduct the initial in vitro biocompatibility assessment. Quantitative LDH measurements showed that pure PGS and the 1-5 wt. % PGS/halloysite nanocomposites were as biocompatible as culture dish material and PDLLA. However, significant cytotoxicity was revealed both in the 10 and 20 wt. % composite (p<0.05). This may be associated with the impact of severe acidity caused by the low crosslink density, reflected by the lower pH values. However, the cytotoxicity detected in the confined culture wells may not exist in vivo, which is an open, constantly flowing system.

Conclusions: In this example we have synthesised and characterized PGS and PGS/halloysite composites, incorporating 1, 3, 5, 10 and 20 wt. % halloysite, with a goal of improving materials' stability while maintaining their flexibility. The studies have found that the addition of nanotubular halloysite has two opposite effects on the PGS elastomeric network. First, the acidic outer layer of halloysite nanotubes reduces crosslink density in the PGS matrix and as a result weakens the network. Second, the PGs macromolecules absorb onto the surface of halloysite tubes, forming a bound rubber and thus strengthening the elastomeric network. The above two opposite effects work together, leading to a satisfactory balance of the degradation and flexibility that cannot be achieved in the polymer alone. Among the six investigated materials (0-20 wt %), the composites of 3 and 5 wt % are the most promising ones, with well retained compliance compared with the pure PGS counterpart, reduced degradation rates, excellent resilience, and satisfactory biocompatibility in vitro.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A crosslinked polyol-bioceramic composite which comprises:
(A) a polymer matrix formed from the condensation reaction between (I) a polyol component containing at least three hydroxyl groups; (II) a polycarboxylic acid component containing at least two carboxylic groups; and
(B) at least one bioceramic material phase substantially homogeneously distributed throughout the polymer matrix;
wherein the amount bioceramic material in the composite is from about 0.5% to about 20% by weight of the total weight of the composite.

2. The composite of claim 1, wherein the amount of bioceramic material in the composite is from about 5% to about 15% by weight of the total weight of the composite.

3. The composite of claim 1, wherein the amount of bioceramic material in the composite is from about 10% by weight of the total weight of the composite.

4. The composite of claim 1, wherein the polyol component is selected from the group consisting of glycerol, erythritol, threitol, ribitol, arabinitol, xylitol, allitol, alritol, galactitol, sorbitol, mannitol, iditol and malitol.

5. The composite of claim 1, wherein the polycarboxylic acid component is an aldaric acid selected from the group consisting of 2-hydroxy-malonic acid, tartaric acid, ribaric acid, arabanaric acid, xylaric acid, aldaric acid, altraric acid, galacteric acid, glucaric acid, mannaric acid, and derivatives and salts thereof.

6. The composite of claim 1, wherein the polycarboxylic acid component is a metabolite selected from the group consisting of succinic acid, fumaric acid, α-ketoglutaric acid, oxaloacetic acid, malic acid, oxalosuccinic acid, isocitric acid, cis-aconitic acid, citric acid, and derivatives and salts thereof.

7. The composite of claim 1, wherein the polycarboxylic acid component is an alkanedioic acid selected from the group consisting of dimercaptosuccinic acid, oxalic acid, malonic acid, succinic acid, glutaric acid, adipic acid, pimelic acid, suberic acid, azelaic acid, sebacic acid, and derivatives and salts thereof.

8. The composite of claim 1, wherein the polycarboxylic acid component is an alkenedioic acid selected from the group consisting of fumaric acid, maleic acid, glutaconic acid, itaconic acid, mesaconic acid, traumatic acid, and derivatives and salts thereof.

9. The composite of claim 1, wherein the amino acid is a member selected from the group consisting of aspartic acid, glutamic acid, and derivatives and salts of aspartic acid and glutamic acid.

10. The composite of claim 1, wherein the at least one bioceramic is selected from the group consisting of alumina, aluminosilicate, zirconia, apatites, calcium phosphates, silica based glasses, and bioactive glass ceramics and combinations and modified forms.

11. The composite of claim 1, wherein the at least one bioceramic is an apatite selected from the group consisting of hydroxyapatite (Ca₁₀(PO₄)₆(OH)₂), floroapatite (Ca₁₀(PO₄)₆F₂), chlorapatite (Ca₅Cl(PO₄)₃), carbonate apatide (Ca₁₀H₂(PO₄)₆-5H₂O)) and combinations and modified forms thereof.

12. The composite of claim 1, wherein the at least one bioceramic is a bioactive glass selected from the group consisting of 45S5, 58S, S53P4, S70C30 and combinations and modified forms thereof.

13. A method of preparing a crosslinked polyol-bioceramic composite, the method comprising the steps of:
(i) providing at least one polyol component containing at least three hydroxyl groups;
(ii) providing at least one polycarboxylic acid component containing at least two carboxylic acid;
(iii) partially reacting the polyol with the polycarboxylic acid to form a prepolymer solution;
(iv) substantially homogeneously distributing at least one bioceramic material throughout the prepolymer solution; and
(v) subjecting the prepolymer solution of step (iv) to further reaction conditions to introduce further crosslinking to form the crosslinked polyol-bioceramic composite.

14. A method of treating a disease, condition, or disorder from which a subject is suffering, comprising administering to the subject a polyol-bioceramic composite of claim 1.

15. A crosslinked polyol-bioceramic composite of claim 1, wherein the polyol-bioceramic composite is adapted and constructed to have a shape selected from the group consisting of particles, tube, sphere, strand, coiled strand, capillary network, film, fiber, mesh and sheet.

16. (canceled)

17. A crosslinked polyol-bioceramic scaffold composite comprising
(A) a porous bioceramic foam formed from at least one bioceramic material; and
(B) a polyol polymer matrix wherein the polyol polymer matrix is formed in situ in the foam by the condensation reaction of (I) a polyol component containing at least three hydroxyl groups; (II) a polycarboxylic acid component containing at least two carboxylic groups;
wherein the amount bioceramic material in the polyol-bioceramic scaffold composite is from about 50% to about 70% by weight of the total weight of the polyol-bioceramic scaffold composite.

18. The polyol-bioceramic scaffold composite of claim 17, wherein the amount of bioceramic material is about 70% by weight of the total weight of the polyol-bioceramic scaffold composite.

19. The polyol-bioceramic scaffold composite of claim 17, wherein the bioceramic is a member selected from the group consisting of alumina, aluminosilicate, zirconia, apatites, calcium phosphates, silica based glasses, and bioactive glass ceramics and combinations and modified forms thereof.

20. The polyol-bioceramic scaffold composite of claim 17, wherein the polyol component is a member selected from the group consisting of glycerol, erythritol, threitol, ribitol, arabinitol, xylitol, allitol, alritol, galactitol, sorbitol, mannitol, iditol and malitol.

21. The polyol-bioceramic scaffold composite of claim 17, wherein the polycarboxylic acid component is an alkenedioic acid selected from the group consisting of fumaric acid, maleic acid, glutaconic acid, itaconic acid, mesaconic acid, or traumatic acid, and derivatives and salts thereof.

22-23. (canceled)

24. A method for promoting tissue growth in a subject suffering from diseased or damaged tissue, said method comprising implanting or injecting a crosslinked polyol-ceramic composite of claim 1 into said subject on or near said diseased or damaged tissue.

25. A method for promoting nerve growth in a subject in need thereof, said method comprising implanting a conduit of a crosslinked polyol-ceramic composite of claim 1 into said subject at a site where such growth is sought.

26. A method for repairing an abdominal hernia in a subject suffering from such a hernia, said method comprising implanting or injecting a crosslinked polyol-ceramic composite of claim 1 into said subject at the site of said hernia.

27. A method for repairing an invertebrate disc in a subject in need of such repair, said method comprising implanting or injecting a crosslinked polyol-ceramic composite of claim 1 into said subject at the site of said disc.

28. A method for correcting a bone defect in a subject suffering from such a defect, said method comprising implanting a crosslinked polyol-ceramic scaffold composite of claim 17 into said subject at the site of said defect.
